SEQUENCE LISTING (38 sequences)

SEQ ID NO: 1 - protein, 86 aa, Homo sapiens
ERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 2 - protein, 286 aa, Homo sapiens
NCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 3 - protein, 195 aa, Homo sapiens
KKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 4 - protein, 234 aa, Artificial Sequence
  spacer sequence, hinge-CH2CH3 of human IgG1
AEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKD

SEQ ID NO: 5 - protein, 46 aa, Artificial Sequence
  spacer sequence, human CD8 stalk
TTTPAPRPPTPAPTIASQPLSLRPEACRPAAGGAVHTRGLDFACDI

SEQ ID NO: 6 - protein, 20 aa, Artificial Sequence
  spacer sequence, human IgG1 hinge
AEPKSPDKTHTCPPCPKDPK

SEQ ID NO: 7 - protein, 21 aa, Homo sapiens
VVISVGSMGLIISLLCVYFWL

SEQ ID NO: 8 - protein, 25 aa, Homo sapiens
IPWLGHLLVGLSGAFGFIILVYLLI

SEQ ID NO: 9 - protein, 25 aa, Homo sapiens
PILLTISILSFFSVALLVILACVLW

SEQ ID NO: 10 - protein, 22 aa, Homo sapiens
AISTSTVLLCGLSAVSLLACYL

SEQ ID NO: 11 - protein, 238 aa, Artificial Sequence
  scFv based on J591
EVQLQQSGPELKKPGTSVRISCKTSGYTFTEYTIHWVKQSHGKSLEWIGNINPNNGGTTYNQKFEDKATLTVDKSSSTAYMELRSLTSEDSAVYYCAAGWNFDYWGQGTTLTVSSGGGGSGGGGSGGGGSDIVMTQSHKFMSTSVGDRVSIICKASQDVGTAVDWYQQKPGQSPKLLIYWASTRHTGVPDRFTGSGSGTDFTLTITNVQSEDLADYFCQQYNSYPLTFGAGTMLDLKR

SEQ ID NO: 12 - protein, 21 aa, Artificial Sequence
  signal peptide
MGTSLLCWMALCLLGADHADG

SEQ ID NO: 13 - protein, 21 aa, Artificial Sequence
  signal peptide
MSLPVTALLLPLALLLHAARP

SEQ ID NO: 14 - protein, 20 aa, Artificial Sequence
  signal peptide
MAVPTQVLGLLLLWLTDARC

SEQ ID NO: 15 - protein, 112 aa, Artificial Sequence
  CD3 Z endodomain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - protein, 152 aa, Artificial Sequence
  CD28 and CD3 Zeta endodomains
SKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - protein, 188 aa, Artificial Sequence
  CD28,OX40 and CD3 Zeta endodomains
SKRSRLLHSDYMNMTPRRPGPTRKHYQPYAPPRDFAAYRSRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 18 - protein, 38 aa, Artificial Sequence
  ICOS endodomain
CWLTKKKYSSSVHDPNGEYMFMRAVNTAKKSRLTDVTL

SEQ ID NO: 19 - protein, 48 aa, Artificial Sequence
  CD27 endodomain
QRRKYRSNKGESPVEPAEPCHYSCPREEEGSTIPIQEDYRKPEPACSP

SEQ ID NO: 20 - protein, 111 aa, Artificial Sequence
  BTLA endodomain
RRHQGKQNELSDTAGREINLVDAHLKSEQTEASTRQNSQVLLSETGIYDNDPDLCFRMQEGSEVYSNPCLEENKPGIVYASLNHSVIGPNSRLARNVKEAPTEYASICVRS

SEQ ID NO: 21 - protein, 188 aa, Artificial Sequence
  CD30 endodomain
HRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 22 - protein, 58 aa, Artificial Sequence
  GITR endodomain
QLGLHIWQLRSQCMWPRETQLLLEVPPSTEDARSCQFPEEERGERSAEEKGRLGDLWV

SEQ ID NO: 23 - protein, 60 aa, Artificial Sequence
  HVEM endodomain
CVKRRKPRGDVVKVIVSVQRKRQEAEGEATVIEALQAPPDVTTVAVEETIPSFTGRSPNH

SEQ ID NO: 24 - protein, 20 aa, Artificial Sequence
  cleavage site, 2A-like sequence
YHADYYKQRLIHDVEMNPGP

SEQ ID NO: 25 - protein, 20 aa, Artificial Sequence
  cleavage site, 2A-like sequence
HYAGYFADLLIHDIETNPGP

SEQ ID NO: 26 - protein, 20 aa, Artificial Sequence
  cleavage site, 2A-like sequence
QCTNYALLKLAGDVESNPGP

SEQ ID NO: 27 - protein, 19 aa, Artificial Sequence
  cleavage site, 2A-like sequence
ATNFSLLKQAGDVEENPGP

SEQ ID NO: 28 - protein, 19 aa, Artificial Sequence
  cleavage site, 2A-like sequence
AARQMLLLLSGDVETNPGP

SEQ ID NO: 29 - protein, 20 aa, Artificial Sequence
  cleavage site, 2A-like sequence
RAEGRGSLLTCGDVEENPGP

SEQ ID NO: 30 - protein, 20 aa, Artificial Sequence
  cleavage site, 2A-like sequence
TRAEIEDELIRAGIESNPGP

SEQ ID NO: 31 - protein, 20 aa, Artificial Sequence
  cleavage site, 2A-like sequence
TRAEIEDELIRADIESNPGP

SEQ ID NO: 32 - protein, 20 aa, Artificial Sequence
  cleavage site, 2A-like sequence
AKFQIDKILISGDVELNPGP

SEQ ID NO: 33 - protein, 20 aa, Artificial Sequence
  cleavage site, 2A-like sequence
SSIIRTKMLVSGDVEENPGP

SEQ ID NO: 34 - protein, 20 aa, Artificial Sequence
  cleavage site, 2A-like sequence
CDAQRQKLLLSGDIEQNPGP

SEQ ID NO: 35 - protein, 20 aa, Artificial Sequence
  cleavage site, 2A-like sequence
YPIDFGGFLVKADSEFNPGP

SEQ ID NO: 36 - protein, 5 aa, Artificial Sequence
  Type I cytokine receptor common motif
  misc_feature (3)..(3): Xaa can be any naturally occurring amino acid
WSXWS

SEQ ID NO: 37 - protein, 4 aa, Artificial Sequence
  basic amino acid furin target sequence
  misc_feature (2)..(2): Xaa can be any naturally occurring amino acid
  MISC_FEATURE (3)..(3): Xaa may be Arg or Lys
RXXR

SEQ ID NO: 38 - protein, 7 aa, Artificial Sequence
  consensus Tobacco Etch Virus (TEV) cleavage site
ENLYFQS

(19) United States
(12) Patent Application Publication
(10) Pub. No.: US 2020/0239545 A1
(43) Pub. Date: Jul. 30, 2020

(54) RECEPTOR

(71) Applicant: AUTOLUS LIMITED, London (GB)

(72) Inventors: Vijay Peddareddigari, London (GB); Martin Pule, London (GB); Simon Thomas, London (GB)

(21) Appl. No.: 16/486,450

(22) PCT Filed: Feb. 16, 2018
(86) PCT No.: PCT/GB2018/050410

§ 371 Date: Aug. 15, 2019

(30) Foreign Application Priority Data
Feb. 17, 2017 (GB) 1702617.0

(51) Int. Cl. C07K 14/715; C12N 5/0783; A61K 35/17

(52) U.S. Cl. CPC C07K 14/7155; C12N 5/0636; C07K 2319/30; C07K 2319/03; C07K 2319/74; A61K 35/17

(57) ABSTRACT

The present invention provides a chimeric cytokine receptor (CCR) comprising: (i) an exodomain which binds to an intracellular ligand which is released from a cell as a result of necrosis; and (ii) a cytokine receptor endodomain.

### FIELD OF THE INVENTION

The present invention relates to a chimeric cytokine receptor (CCR), and a cell which expresses such a chimeric cytokine receptor and optionally a chimeric antigen receptor at the cell surface.

### BACKGROUND TO THE INVENTION

### Chimeric Antigen Receptors (CARs)

A number of immunotherapeutic agents have been described for use in cancer treatment, including therapeutic monoclonal antibodies (mAbs), bi-specific T-cell engagers and chimeric antigen receptors (CARs).

Chimeric antigen receptors are proteins which graft the specificity of a monoclonal antibody (mAb) to the effector function of a T-cell. Their usual form is that of a type I transmembrane domain protein with an antigen recognizing amino terminus, a spacer, a transmembrane domain all connected to a compound endodomain which transmits T-cell survival and activation signals.

The most common form of these molecules are fusions of single-chain variable fragments (scFv) derived from monoclonal antibodies which recognize a target antigen, fused via a spacer and a trans-membrane domain to a signaling endodomain. Such molecules result in activation of the T-cell in response to recognition by the scFv of its target. When T cells express such a CAR, they recognize and kill target cells that express the target antigen. Several CARs have been developed against tumour associated antigens, and adoptive transfer approaches using such CAR-expressing T cells are currently in clinical trial for the treatment of various cancers.

### CAR-Based Approaches to Treat Solid Cancers

Several case series have reported clinically significant responses in patients with CD19-positive haematological malignancies. However clinical experience targeting solid tumour antigens with CAR T-cells is considerably more limited. Some of the key issues appear to be the absence of unique tumour-associated antigens, the inefficient homing of T-cells to tumour sites and the failure of CAR T-cells to engraft and expand within a solid cancer tumour bed.

CAR T-cell persistence and activity can be enhanced by administration of cytokines, or by the CAR T-cells producing cytokines constitutively. However, these approaches have limitations: systemic administration of cytokines can be toxic; constitutive production of cytokines may lead to uncontrolled proliferation and transformation (Nagarkatti et al (1994) PNAS 91:7638-7642; Hassuneh et al (1997) Blood 89:610-620).

There is therefore a need for alternative CAR T-cell approaches, which facilitate engraftment and expansion of T cells to counteract the effects of the hostile tumour microenvironment.

### On-Target Off-Tumour Toxicity

It is relatively rare for the presence of a single antigen effectively to describe a cancer, which can lead to a lack of specificity.

Most cancers cannot be differentiated from normal tissues on the basis of a single antigen. Hence, considerable “on-target off-tumour” toxicity occurs whereby normal tissues are damaged by the therapy. For instance, whilst targeting CD20 to treat B-cell lymphomas with Rituximab, the entire normal B-cell compartment is depleted, whilst targeting CD52 to treat chronic lymphocytic leukaemia, the entire lymphoid compartment is depleted, whilst targeting CD33 to treat acute myeloid leukaemia, the entire myeloid compartment is damaged etc.

The predicted problem of “on-target off-tumour” toxicity has been borne out by clinical trials. For example, an approach targeting ERBB2 caused death to a patient with colon cancer metastatic to the lungs and liver. ERBB2 is over-expressed in colon cancer in some patients, but it is also expressed on several normal tissues, including heart and normal vasculature.

There is therefore a need for improved approaches to cancer therapy in which such “on-target off-tumour” toxicity is reduced or eliminated.

### DESCRIPTION OF THE FIGURES

FIG. 1: Schematic diagram summarising the structure of various cytokine receptors, the cell types which produce the cytokines and the cell types which express the cytokine receptors.

FIG. 2: Schematic diagram showing proposed chimeric cytokine receptor

(a) Cytokine IL2 and IL7 cytokine receptors signal through a common gamma chain and a cytokine specific alpha/beta chain.

(b) One implementation of a chimeric cytokine receptor is to replace the ectodomain of the cytokine alpha/beta and gamma chain with different scFvs (or any other suitable binder) which recognize different epitopes of PSA.

(c) An alternative approach is to replace the ectodomains of alpha/beta and gamma with the VH/VL of a PSA specific antibody, where both VH and VL are involved in binding so that binding brings them together.

FIG. 3: Aggregation-based cytokine signalling enhancer Schematic diagram showing a chimeric cytokine receptor and CAR combination system. The cell comprises two chimeric cytokine receptors which bind different epitopes on the same soluble ligand. In the absence of soluble ligand (e.g. PSA) but the presence of the cell-membrane antigen (e.g. PSMA) signalling occurs thought the CAR. In the presence of the soluble ligand, aggregation of the two chimeric cytokine receptors occurs, leading to cytokine-based signal enhancement.

FIG. 4: Theoretical construct map for the chimeric cytokine receptor/CAR combination system illustrated in FIG. 3.

FIG. 5: Schematic diagram illustrating an example of a structure for the chimeric transmembrane protein of the present invention. The chimeric transmembrane protein comprises a dimerization domain and a cytokine receptor endodomain. The embodiment shown has a “Fab” type architecture, as the dimerization domain comprises antibody-type heavy and light chain constant regions. Constant dimerization between these domains brings together the IL2 receptor common γ chain with either the IL-2 receptor β chain or the IL-7 receptor α chain, leading to constitutive cytokine signalling.

FIG. 6: IL-2 signalling by the chimeric transmembrane protein.

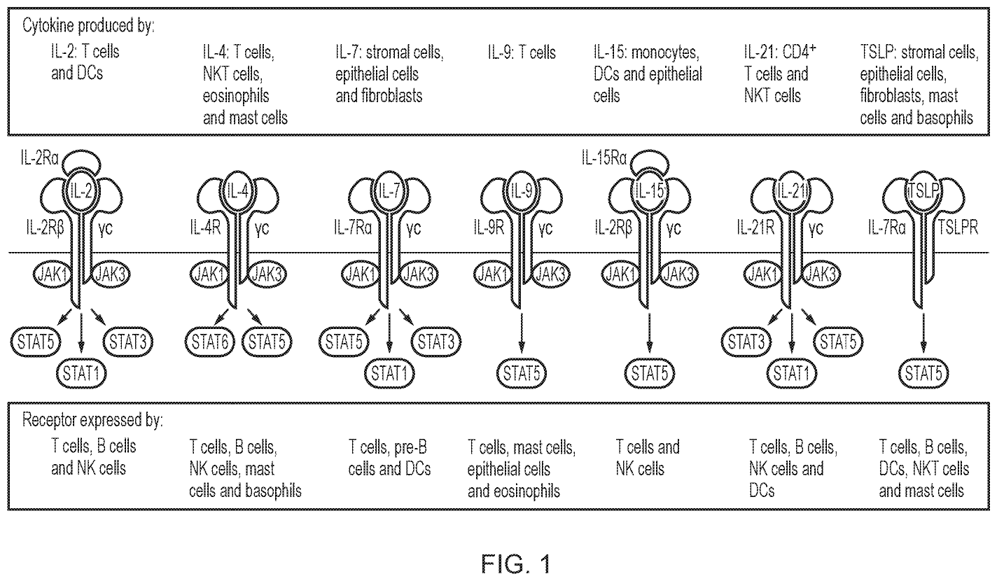

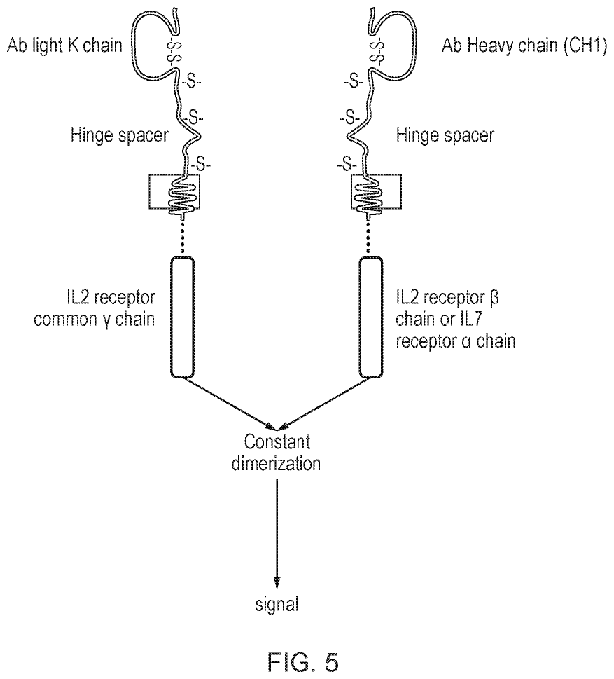

Two chimeric transmembrane proteins having the general structure shown in FIG. 5 were tested for their ability to induce IL-2 signalling. One chimeric transmembrane protein comprised an IL2 receptor endodomain and the other comprised an IL-7 receptor endodomain. IL-2 signalling was tested using the murine cell line CTLL2 which is dependent on IL-2 signalling for growth. As a positive control, CTLL2 cells were cultured with 100 u/mL murine IL2. Cells expressing the chimeric transmembrane protein comprising the IL2 receptor endodomain (Fab_IL2endo) supported CTLL2 cell survival and growth, whereas cells expressing the chimeric transmembrane protein comprising the IL-7 receptor (Fab_IL7endo) did not.

FIG. 7: Schematic diagram illustrating panel of PSA chimeric cytokine receptors

A panel of chimeric cytokine receptors (CCRs) targeting PSA was developed using scFvs derived from two antibodies which bind to different PSA epitopes: 5D5A5 and 5D3D11.

Top-left panel: A CCR with an IL-2R endodomain having A5 on the chain with IL2R β chain and D11 on the chain with common γ chain;

Top-right panel: A CCR with an IL7R endodomain having A5 on the chain with IL7R α chain and D11 on the chain with common γ chain;

Bottom-left panel: A CCR with an IL-2R endodomain having D11 on the chain with IL2R β chain and A5 on the chain with common γ chain; and

Bottom-right hand panel: A CCR with an IL-7R endodomain having D11 on the chain with IL7R α chain and A5 on the chain with common γ chain.

A negative control was also created for each CCR, in which the IL2Rγ chain was replaced by a rigid linker.

FIG. 8: IL2 signalling from cells expressing a PSA chimeric cytokine receptor in the presence of PSA-CTLL2 proliferation

CTLL2 cells were transduced with constructs expressing some of the PSA chimeric cytokine receptors illustrated in FIG. 7. Cells were cultured in the presence of absence of IL2 (positive control) and the presence of absence of 5 ng/mL or 5 μg/mL PSA. CTLL2 proliferation was assessed after 3 and 7 days.

The anti-PSA chimeric cytokine receptor with an IL2R endodomain supported CTLL2 cell proliferation in the absence of IL2 and the presence of PSA, but not the receptor having an IL7R endodomain or any of the CCRs comprising a rigid linker in the place of the common γ chain.

FIG. 9: IL2 signalling from cells expressing a PSA chimeric cytokine receptor in the presence of PSA-CTLL2 STAT5 phosphorylation

CTLL2 cells were either left untransduced (WT); or transduced with a vector expressing a CCR against PSA (D11-CD8STK-IL2Rg_A5-Hinge-IL2Rb) or an equivalent construct having a rigid linker in the place of the common γ chain (D11-CD8STK-RL_A5-Hinge-IL2Rb). Cells were incubated with either 500 μM Pervanadate or 500 ng/mL PSA for 1 or 4 hours. Phosphorylation of Y694 of STAT5 was then investigated using phosphoflow.

### SUMMARY OF ASPECTS OF THE INVENTION

The present inventors have previously developed “chimeric cytokine receptors” (CCR) which graft the binding specificity of a non-cytokine binding molecule on to the endodomain of a cytokine receptor. The co-expression of such a CCR with a chimeric antigen receptor (CAR) helps a CAR T-cell to engraft and expand in the hostile tumour microenvironment. The requirement for the ligand for the CCR as well as the ligand for the CAR to be present add another layer of selectivity and helps prevent on-target off-tumour toxicity.

For example, they have previously described a cell which co-expresses a CAR with a chimeric cytokine receptor which detects PSA and transmits an IL2/15 or an IL7 signal to the CAR T-cell. In this way, the CAR T-cell is stimulated to proliferate selectively only in a prostate cancer microenvironment, and in the absence of PSA (i.e. after the patient is in remission), the cytokine stimulation is lost.

The same technology can be used to detect another source of tumour-specific ligands, namely antigens which are released from cells during the process of necrosis.

Necrosis is a characteristic of solid tumours, resulting in part from prolonged hypoxia. Apoptosis and necrosis are two important mechanisms of cell death. Apoptosis is programmed cell death, whereas necrosis is commonly viewed as “unprogrammed”. Apoptosis results in the formation of membrane-enclosed apoptotic bodies that contain intact organelles and cytoplasm which are engulfed by phagocytic cells. Necrotic cells are characterised by organelle swelling, nuclear condensation and rupture of the plasma membrane. Apoptosis is a “clean” form of cell death in which very little inflammatory activation occurs. In contrast, necrosis is a “messy” from of death, releasing factors into the extracellular milieu which results in activation of the inflammatory response.

The present invention involves making use of the factors released from necrotic cells in a tumour to stimulate CAR T-cells.

In a first aspect, the present invention provides a chimeric cytokine receptor (CCR) comprising:
an exodomain which binds to a ligand an intracellular ligand which is released from a cell as a result of necrosis; and
a cytokine receptor endodomain.

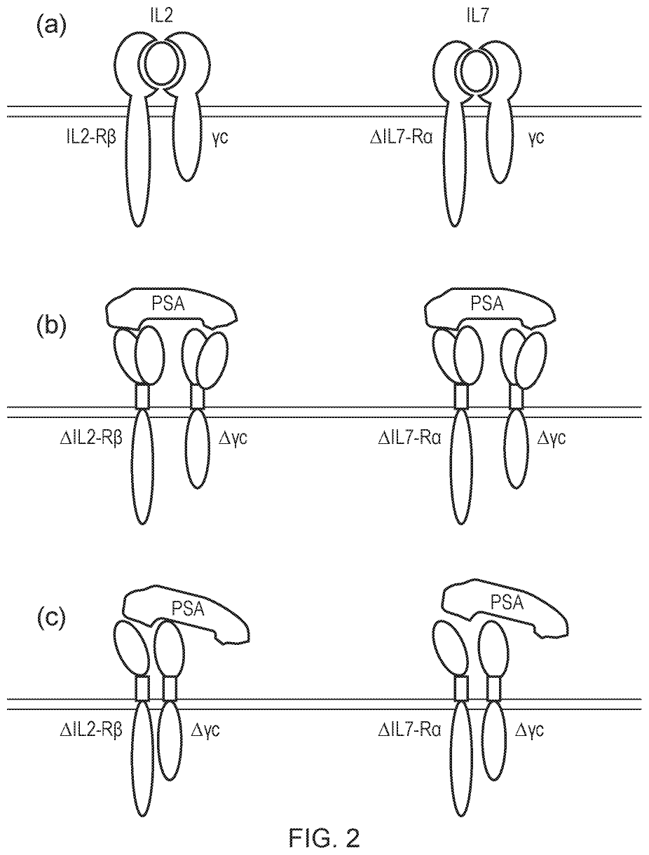

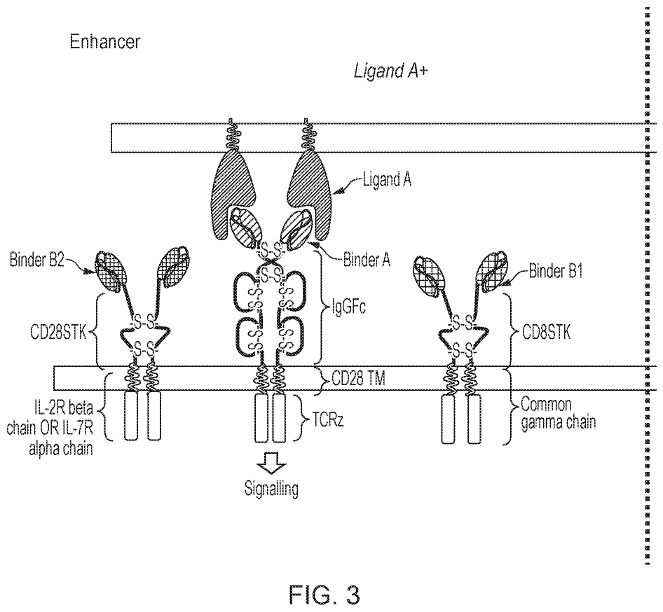

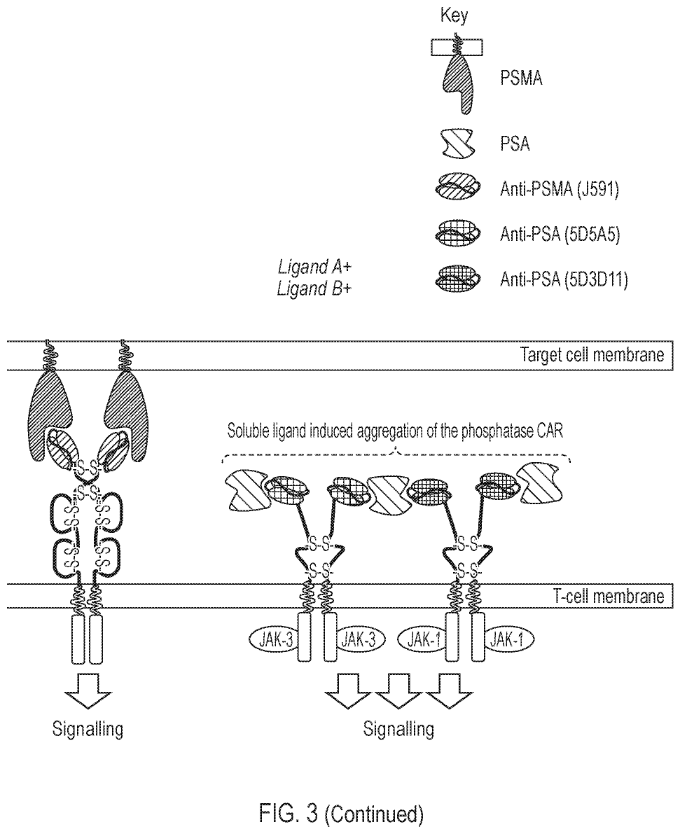

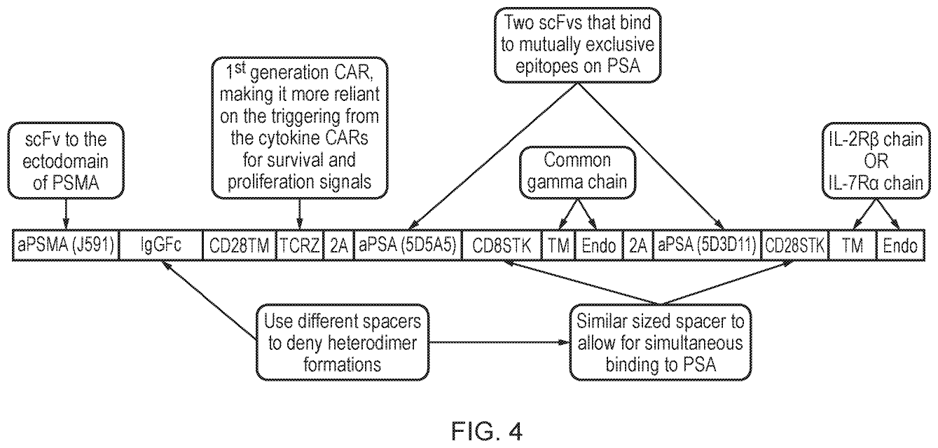

In a first embodiment of the first aspect of the invention, the chimeric cytokine receptor comprises two polypeptides:
(i) a first polypeptide which comprises:
   (a) a first antigen-binding domain which binds a first epitope of the ligand
   (b) a first chain of the cytokine receptor endodomain; and
(ii) a second polypeptide which comprises:
   (a) a second antigen-binding domain which binds a second epitope of the ligand
   (b) a second chain of the cytokine-receptor endodomain. FIG. 2b illustrates such an arrangement.

Each of the first and second antigen-binding domains may be, for example, single-chain variable fragments (scFvs) or single domain binders.

In a second embodiment of the first aspect of the invention, the chimeric cytokine receptor which comprises two polypeptides:
(i) a first polypeptide which comprises:
   (a) a heavy chain variable domain (VH)
   (b) a first chain of the cytokine receptor endodomain; and
(ii) a second polypeptide which comprises:
   (a) a light chain variable domain (VL)
   (b) a second chain of the cytokine-receptor endodomain.

FIG. 2c illustrates such an arrangement.

The first and second chains for the cytokine receptor endodomains may be different and may be selected from type I cytokine receptor endodomain α-, β-, and γ-chains.

Alternatively the first and second chains for the cytokine receptor endodomains may be the same and may be selected from type I cytokine receptor endodomain α-, β-, and γ-chains.

For example, the cytokine receptor endodomain may comprise:
(i) IL-2 receptor β-chain endodomain
(ii) IL-7 receptor α-chain endodomain;
(iii) IL-15 receptor α-chain endodomain; or
(iv) common γ-chain receptor endodomain.

The cytokine receptor endodomain may comprise (i), (ii) or (iii); and (iv).

The ligand may be an intracellular molecule which is not expressed at the cell surface or secreted by the cell. The ligand may be a molecule which is located intracellularly in a healthy cell, but is released from the cell during necrosis into the extracellular milieu. It may be released from the cell as a result of rupture of the plasma membrane. It may be released from an organelle or expressed on an organelle membrane.

The ligand may be normally (i.e. in a normal, non-necrotic cell) located in an intracellular location such as the cytosol, nucleus or an organelle of the non-necrotic cell.

In a second aspect, the present invention provides a cell which comprises a chimeric cytokine receptor according to the first aspect of the invention.

The cell may comprise a first chimeric cytokine receptor and a second chimeric cytokine receptor which bind different epitopes on the same ligand.

The cell may comprise a first chimeric cytokine receptor which comprises a type I cytokine receptor endodomain α- or β-chain, and a second chimeric cytokine receptor which comprises a type I cytokine receptor endodomain γ-chain, such that when the first chimeric cytokine receptor and the second cytokine receptor bind the ligand, combined signalling through the α-/β-chain and γ-chain occurs.

The cell may also comprise a chimeric antigen receptor, for example a chimeric antigen receptor which binds a tumour-associated cell surface antigen.

The chimeric antigen receptor may bind a cell surface antigen associated with a solid tumour. For example, the CAR may bind may bind a cell surface antigen associated with breast, prostate, lung, pancreas or colon cancer. The CAR may bind may bind a cell surface antigen associated with prostate cancer, such as prostate stem-cell antigen (PSCA) or prostate-specific membrane antigen (PSMA).

In a third aspect, the present invention provides a nucleic acid sequence encoding a chimeric cytokine receptor (CCR) according to the first aspect of the invention.

In a fourth aspect the present invention provides a nucleic acid construct which comprises a first nucleic acid sequence encoding a first CCR and a second nucleic acid sequence encoding a second CCR, the nucleic acid construct having the structure:
AgB1-spacer1-TM1-endo1-coexpr-AbB2-spacer2-TM2-endo2
in which
AgB1 is a nucleic acid sequence encoding the antigen-binding domain of the first CCR;
spacer 1 is a nucleic acid sequence encoding the spacer of the first CCR;
TM1 is a nucleic acid sequence encoding the transmembrane domain of the first CCR;
endo 1 is a nucleic acid sequence encoding the endodomain of the first CCR;
coexpr is a nucleic acid sequence enabling co-expression of both CCRs
AgB2 is a nucleic acid sequence encoding the antigen-binding domain of the second CCR;
spacer 2 is a nucleic acid sequence encoding the spacer of the second CCR;
TM2 is a nucleic acid sequence encoding the transmembrane domain of the second CCR;
endo 2 is a nucleic acid sequence encoding the endodomain of the second CCR.

The nucleic acid construct may also encode a chimeric antigen receptor (CAR). In this embodiment, the nucleic acid construct may have the structure:
(i) CCRAgB1-CCRspacer1-CCRTM1-CCRendo1-coexpr1-CCRAgB2-CCRspacer2-CCRTM2-CCRendo2-coexpr2-CARAgB-CARspacer-CARTM-CARendo;
(ii) CCRAgB1-CCRspacer1-CCRTM1-CCRendo1-coexpr1-CARAgB-CARspacer-CARTM-CARendo-coexpr2-CCRAgB2-CCRspacer2-CCRTM2-CCRendo2; or
(iii) CARAgB-CARspacer-CARTM-CARendo-coexpr1-CCRAgB1-CCRspacer1-CCRTM1-CCRendo1-coexpr2-CCRAgB2-CCRspacer2-CCRTM2-CCRendo2;
in which
CCRAgB1 is a nucleic acid sequence encoding the antigen-binding domain of the first CCR;
CCRspacer1 is a nucleic acid sequence encoding the spacer of the first CCR;
CCRTM1 is a nucleic acid sequence encoding the transmembrane domain of the first CCR;
CCRendo1 is a nucleic acid sequence encoding the endodomain of the first CCR;
CCRAgB2 is a nucleic acid sequence encoding the antigen-binding domain of the second CCR;
CCRspacer2 is a nucleic acid sequence encoding the spacer of the second CCR;
CCRTM2 is a nucleic acid sequence encoding the transmembrane domain of the second CCR;
CCRendo2 is a nucleic acid sequence encoding the endodomain of the second CCR;
Coexpr1 and coexpr2 are nucleic acid sequences enabling co-expression of the two flanking sequences;
CARAgB is a nucleic acid sequence encoding the antigen-binding domain of the CAR;
CARspacer is a nucleic acid sequence encoding the spacer of the CAR;
CARTM is a nucleic acid sequence encoding the transmembrane domain of the CAR; and
CARendo is a nucleic acid sequence encoding the endodomain of the CAR.

Any or all of the sequences coexpr, coexpr1, coexpr2 may encode a sequence comprising a self-cleaving peptide.

Alternative codons may be used in regions of sequence encoding the same or similar amino acid sequences, in order to avoid homologous recombination.

In a fifth aspect, the present invention provides a vector comprising a nucleic acid construct according to the fourth aspect of the invention.

The vector may be, for example, a retroviral vector or a lentiviral vector or a transposon.

In a sixth aspect, the present invention provides a kit which comprises:
i) a vector comprising a nucleic acid sequence encoding a first CCR according to the first aspect of the invention; and
ii) a vector comprising a nucleic acid sequence encoding a second CCR according to the second aspect of the invention.

The kit may also comprise a vector comprising a nucleic acid sequence encoding a chimeric antigen receptor.

The kit may comprise:
i) a vector comprising a nucleic acid sequence encoding a CCR according to the first aspect of the invention; and
ii) a vector comprising a nucleic acid sequence encoding a chimeric antigen receptor.

In a seventh aspect, the present invention provides a method for making a cell according to the second aspect of the invention, which comprises the step of introducing: a nucleic acid sequence according to the third aspect of the invention; a nucleic acid construct according to the fourth aspect of the invention; a vector according to the fifth aspect of the invention; or a kit of vectors according to the sixth aspect of the invention, into a cell.

The cell may be from a sample isolated from a subject.

In an eighth aspect, there is provided a pharmaceutical composition comprising a plurality of cells according to the second aspect of the invention.

In a ninth aspect, there is provided a method for treating and/or preventing a disease, which comprises the step of administering a pharmaceutical composition according to the eighth aspect of the invention to a subject.

The method may comprise the following steps:
(i) isolation of a cell-containing sample from a subject;
(ii) transduction or transfection of the cells with: a nucleic acid sequence according to the third aspect of the invention; a nucleic acid construct according to the fourth aspect of the invention; a vector according to the fifth aspect of the invention; or a kit of vectors according to the sixth aspect of the invention; and
(iii) administering the cells from (ii) to a the subject.

The sample may be a T-cell containing sample.

The disease may be a cancer, for example a solid cancer such as breast, prostate, lung, pancreas or colon cancer.

There is also provided a pharmaceutical composition according to the eighth aspect of the invention for use in treating and/or preventing a disease.

There is also provided the use of a cell according to the second aspect of the invention in the manufacture of a medicament for treating and/or preventing a disease.

When a cell co-expresses a CAR with a chimeric cytokine receptor which detects a necrotic marker, the presence of the necrotic marker causes the CCR to transmit a cytokine signal, such as an IL2/15 or an IL7 signal to the CAR T-cell. In this way, the CAR T-cell is stimulated to proliferate selectively only in a necrotic microenvironment, which is typical of solid cancers. When the T-cell leaves the dite of necrosis and enters healthy tissue, the cytokine stimulation is lost.

The co-expression of such a CCR with a chimeric antigen receptor (CAR) thus helps a CAR T-cell to engraft and expand in the hostile tumour microenvironment. The requirement for the necrotic marker for the CCR as well as the ligand for the CAR to be present add another layer of selectivity and helps prevent on-target off-tumour toxicity.

### DETAILED DESCRIPTION

### Chimeric Cytokine Receptor (CCR)

A chimeric cytokine receptor (CCR) is a molecule which comprises a cytokine receptor endodomain and a heterologous ligand-binding exodomain. The heterologous exodomain binds a ligand other than the cytokine for which the cytokine receptor from which the endodomain was derived is selective. In this way, it is possible to alter the ligand specificity of a cytokine receptor by grafting on a heterologous binding specificity.

A chimeric cytokine receptor comprises:

(i) a ligand binding exodomain;
(ii) an optional spacer;
(iii) a transmembrane domain; and
(iv) a cytokine-receptor endodomain.

### Cytokine Receptors and Signalling

Many cell functions are regulated by members of the cytokine receptor superfamily. Signalling by these receptors depends upon their association with Janus kinases (JAKs), which couple ligand binding to tyrosine phosphorylation of signalling proteins recruited to the receptor complex. Among these are the signal transducers and activators of transcription (STATs), a family of transcription factors that contribute to the diversity of cytokine responses.

When the chimeric cytokine receptor of the invention binds its ligand, one or more of the following intracellular signaling pathways may be initiated:

(i) the JAK-STAT pathway
(ii) the MAP kinase pathway; and
(iii) the Phosphoinositide 3-kinase (PI3K) pathway.

The JAK-STAT system consists of three main components: (1) a receptor (2) Janus kinase (JAK) and (3) Signal Transducer and Activator of Transcription (STAT).

JAKs, which have tyrosine kinase activity, bind to cell surface cytokine receptors. The binding of the ligand to the receptor triggers activation of JAKs. With increased kinase activity, they phosphorylate tyrosine residues on the receptor and create sites for interaction with proteins that contain phosphotyrosine-binding SH2 domains. STATs possessing SH2 domains capable of binding these phosphotyrosine residues are recruited to the receptors, and are themselves tyrosine-phosphorylated by JAKs. These phosphotyrosines then act as binding sites for SH2 domains of other STATs, mediating their dimerization. Different STATs form hetero- or homodimers. Activated STAT dimers accumulate in the cell nucleus and activate transcription of their target genes.

### Cytokine Receptor Endodomain

The chimeric cytokine receptor of the present invention comprises an endodomain which causes “cytokine-type” cell signalling (either alone or when in the presence of another chimeric cytokine receptor) when the exodomain binds its ligand.

The endodomain may be a cytokine receptor endodomain.

The endodomain may be derived from a type I cytokine receptor. Type I cytokine receptors share a common amino acid motif (WSXWS) in the extracellular portion adjacent to the cell membrane.

The endodomain may be derived from a type II cytokine receptor. Type II cytokine receptors include those that bind type I and type II interferons, and those that bind members of the interleukin-10 family (interleukin-10, interleukin-20 and interleukin-22).

Type I cytokine receptors include:
(i) Interleukin receptors, such as the receptors for IL-2, IL-3, IL-4, IL-5, IL-6, IL-7, IL-9, IL-11, IL-12, IL13, IL-15, IL-21, IL-23 and IL-27;
(ii) Colony stimulating factor receptors, such as the receptors for erythropoietin, GM-CSF, and G-CSF; and
(iii) Hormone receptor/neuropeptide receptor, such as hormone receptor and prolactin receptor

Members of the type I cytokine receptor family comprise different chains, some of which are involved in ligand/cytokine interaction and others that are involved in signal transduction. For example the IL-2 receptor comprises an α-chain, a β-chain and a γ-chain.

The IL-2 receptor common gamma chain (also known as CD132) is shared between the IL-2 receptor, IL-4 receptor, IL-7 receptor, IL-9 receptor, IL-13 receptor and IL-15 receptor.

### IL-2

IL-2 binds to the IL-2 receptor, which has three forms, generated by different combinations of three different proteins, often referred to as “chains”: α, β and γ; these subunits are also parts of receptors for other cytokines. The β and γ chains of the IL-2R are members of the type I cytokine receptor family.

The three receptor chains are expressed separately and differently on various cell types and can assemble in different combinations and orders to generate low, intermediate, and high affinity IL-2 receptors.

The α chain binds IL-2 with low affinity, the combination of β and γ together form a complex that binds IL-2 with intermediate affinity, primarily on memory T cells and NK cells; and all three receptor chains form a complex that binds IL-2 with high affinity (Kd˜10-11 M) on activated T cells and regulatory T cells.

The three IL-2 receptor chains span the cell membrane and extend into the cell, thereby delivering biochemical signals to the cell interior. The alpha chain does not participate in signalling, but the beta chain is complexed with the tyrosine phosphatase JAK1. Similarly the gamma chain complexes with another tyrosine kinase called JAK3. These enzymes are activated by IL-2 binding to the external domains of the IL-2R.

IL-2 signalling promotes the differentiation of T cells into effector T cells and into memory T cells when the initial T cells are also stimulated by an antigen. Through their role in the development of T cell immunologic memory, which depends upon the expansion of the number and function of antigen-selected T cell clones, they also have a key role in long-term cell-mediated immunity.

The chimeric cytokine receptor of the present invention may comprise the IL-2 receptor β-chain and/or the IL-2 receptor (i.e. common) γ-chain

The amino acid sequences for the endodomains of the IL-2 β-chain and common γ-chain are shown as SEQ ID No. 1 and 2

SEQ ID No. 1:
Endodomain derived from human common gamma chain:
ERTMPRIPTLKNLEDLVTEYHGNFSAWSGVSKGLAESLQPDYSERLCLVS
EIPPKGGALGEGPGASPCNQHSPYWAPPCYTLKPET
SEQ ID No. 2:
Endodomain derived from human IL-2Rβ:
NCRNTGPWLKKVLKCNTPDPSKFFSQLSSEHGGDVQKWLSSPFPSSSFSP
GGLAPEISPLEVLERDKVTQLLLQQDKVPEPASLSSNHSLTSCFTNQGYF
FFHLPDALEIEACQVYFTYDPYSEEDPDEGVAGAPTGSSPQPLQPLSGED
DAYCTFPSRDDLLLFSPSLLGGPSPPSTAPGGSGAGEERMPPSLQERVPR
DWDPQPLGPPTPGVPDLVDFQPPPELVLREAGEEVPDAGPREGVSFPWSR
PPGQGEFRALNARLPLNTDAYLSLQELQGQDPTHLV

The term “derived from” means that the endodomain of the chimeric cytokine receptor of the invention has the same sequence as the wild-type sequence of the endogenous molecule, or a variant thereof which retains the ability to form a complex with JAK-1 or JAK-3 and activate one of the signalling pathways mentioned above.

A “variant” sequence having at least 80, 85, 90, 95, 98 or 99% sequence identity to the wild-type sequence (e.g. SEQ ID Nos. 1 or 2), providing that the variant sequence retains the function of the wild-type sequence i.e. the ability to form a complex with JAK-1 or JAK-3 and activate, for example, the JAK-STAT signalling pathway.

The percentage identity between two polypeptide sequences may be readily determined by programs such as BLAST which is freely available at http://blast.ncbi.nlm.nih.gov.

### IL-7

The interleukin-7 receptor is made up of two chains: the interleukin-7 receptor-α chain (CD127) and common-γ chain receptor (CD132). The common-γ chain receptors is shared with various cytokines, including interleukin-2, -4, -9, and -15. Interleukin-7 receptor is expressed on various cell types, including naive and memory T cells.

The interleukin-7 receptor plays a critical role in the development of lymphocytes, especially in V(D)J recombination. IL-7R also controls the accessibility of a region of the genome that contains the T-cell receptor gamma gene, by STAT5 and histone acetylation. Knockout studies in mice suggest that blocking apoptosis is an essential function of this protein during differentiation and activation of T lymphocytes.

The chimeric cytokine receptor of the present invention may comprise the IL-7 receptor α-chain and/or the IL-7 receptor (i.e. common) γ-chain, or a variant thereof.

The amino acid sequence for the endodomain of the IL-7 α-chain is shown as SEQ ID No. 3.

SEQ ID No. 3
Endodomain derived from human IL-7Rα:
KKRIKPIVWPSLPDHKKTLEHLCKKPRKNLNVSFNPESFLDCQIHRVDDI
QARDEVEGFLQDTFPQQLEESEKQRLGGDVQSPNCPSEDVVITPESFGRD
SSLTCLAGNVSACDAPILSSSRSLDCRESGKNGPHVYQDLLLSLGTTNST
LPPPFSLQSGILTLNPVAQGQPILTSLGSNQEEAYVTMSSFYQNQ
IL-15

Interleukin 15 (IL-15) is a cytokine with structural similarity to IL-2. Like IL-2, IL-15 binds to and signals through a complex composed of IL-2/IL-15 receptor beta chain (CD122) and the common gamma chain (gamma-C, CD132). IL-15 is secreted by mononuclear phagocytes (and some other cells) following viral infection. IL-15 induces cell proliferation of natural killer cells.

Interleukin-15 receptor consists of an interleukin 15 receptor alpha subunit and shares common beta and gamma subunits with the IL-2 receptor.

### Spacer

The chimeric cytokine receptor of the present invention may comprise a spacer to connect the antigen-binding domain with the transmembrane domain and spatially separate the antigen-binding domain from the endodomain. A flexible spacer allows to the antigen-binding domain to orient in different directions to enable antigen binding.

Where the cell of the present invention comprises two or more chimeric cytokine receptors, the spacers may be the same or different. Where the cell of the present invention comprises a chimeric cytokine receptor (CCR) and a chimeric antigen receptor (CAR), the spacer of the CCR and the CAR may be different, for example, having a different length. The spacer of the CAR may be longer than the spacer of the or each CCR.

The spacer sequence may, for example, comprise an IgG1 Fc region, an IgG1 hinge or a CD8 stalk. The linker may alternatively comprise an alternative linker sequence which has similar length and/or domain spacing properties as an IgG1 Fc region, an IgG1 hinge or a CD8 stalk.

A human IgG1 spacer may be altered to remove Fc binding motifs.

Examples of amino acid sequences for these spacers are given below:

(hinge-CH2CH3 of human IgG1)
SEQ ID No. 4
AEPKSPDKTHTCPPCPAPPVAGPSVFLFPPKPKDTLMIARTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPGKKD
(human CD8 stalk):
SEQ ID No. 5
TTTPAPRPPTPAPTIASQPLSLRPEACRPAAGGAVHTRGLDFACDI
(human IgG1 hinge):
SEQ ID No. 6
AEPKSPDKTHTCPPCPKDPK

### Transmembrane Domain

The transmembrane domain is the sequence of a CCR that spans the membrane. It may comprise a hydrophobic alpha helix. The transmembrane domain may be derived from CD28, which gives good receptor stability.

Alternatively the transmembrane domain may be derived from a cytokine receptor, for example the same cytokine from which the endodomain is derived.

The transmembrane domain may, for example be derived from IL-2R, IL-7R or IL-15R.

Transmembrane derived from human common
gamma chain:
SEQ ID No. 7
VVISVGSMGLIISLLCVYFWL
Transmembrane derived from human IL-2Rβ:
SEQ ID No. 8
IPWLGHLLVGLSGAFGFIILVYLLI
Transmembrane derived from human IL-7Rα:
SEQ ID No. 9
PILLTISILSFFSVALLVILACVLW
Transmembrane derived from human IL-15Rα:
SEQ ID No. 10
AISTSTVLLCGLSAVSLLACYL

### Ligand-Binding Exodomain

The ligand binding domain comprises an antigen binding domain. The antigen binding domain binds the target ligand for the CCR, i.e. the intracellular ligand which is released from a cell as a result of necrosis.

Numerous antigen-binding domains are known in the art, including those based on the antigen binding site of an antibody, antibody mimetics, and T-cell receptors. For example, the antigen-binding domain may comprise: a single-chain variable fragment (scFv) derived from a monoclonal antibody; the binding domain from a natural receptor for the target antigen; a peptide with sufficient affinity for the target ligand; a single domain binder such as a camelid; an artificial binder single as a Darpin; or a single-chain derived from a T-cell receptor.

The term “ligand” is used synonymously with “antigen” to mean an entity which is specifically recognised and bound by the antigen-binding domain of the CCR.

### Ligand

The CCR of the present invention binds a ligand.

The ligand may be released from a cell as a result of necrosis of the cell.

Necrosis is a form of cell injury which results in the premature death of cells in living tissue by autolysis. Necrosis is caused by factors external to the cell or tissue, such as infection, toxins, trauma, hypoxia or ischemia i.e. lack of blood flow. It results in the unregulated digestion of cell components.

Cellular death due to necrosis does not follow the apoptotic signal transduction pathway, but rather various receptors are activated, and result in the loss of cell membrane integrity and an uncontrolled release of products of cell death into the extracellular space. This initiates an inflammatory response in the surrounding tissue which prevents nearby phagocytes from locating and eliminating the dead cells by phagocytosis.

The ligand may be any cell component or cell product released as a result of necrosis. Where necrosis is a marker for diseased tissue, such as cancerous tissue, it is possible to use a cell component or cell product released as a result of necrosis to selectively activate a cell, such as an autologous T cell, in the relevant area of the body. In order to achieve this selectivity, a ligand should be chosen which is released as a result of necrosis but not found to any significant extent on healthy cells or in the extracellular milieu of healthy tissue.

The ligand may be any type of molecule, such as a protein, carbohydrate, nucleic acid or lipid.

The ligand may be “intracellular” in the sense that in a healthy cell it is not visible to a ligand-specific CCR. This may be because in a healthy cell the ligand is located inside the cell and not expressed at the cell surface or secreted by the cell at a level to cause significant CCR-based cytokine stimulation.

When the cell undergoes necrosis, the ligand is released from the cell, such that it may be recognised by a ligand-specific CCR. The local concentration of the ligand released from the cell during necrosis is sufficient to cause CCR-based cytokine stimulation.

The ligand may be a soluble ligand such as a soluble cytosolic protein or other molecule.

Alternatively, the ligand may be a membrane bound ligand, such as a membrane-bound antigen from an organelle. The ligand may be bound to the plasma membrane on the inner leaflet, so that it is not “visible” to a CCR when the cell is intact but during necrosis, the plasma membrane is disrupted revealing the intracellular side of the plasma membrane to CCR-expressing cells.

The ligand may be released from the cytoplasm or the nucleus of a cell. The ligand may be derivable from a cell organelle, such as a mitochondrion, the endoplasmic reticulum, the Golgi apparatus, lysosome, peroxisome or vacuole.

The ligand may be or comprise a nucleic acid sequence. For example the ligand may be or comprise double stranded DNA (dsDNA). The release of dsDNA is characteristic of many tumours.

The ligand may be released from a cancer cell during necrosis. For example, the ligand may be released from a breast, prostate, lung, pancreas or colon cancer cell during necrosis. The ligand may be characteristic of the cancer cell type from which it is released, for example, the ligand may be released from a breast, prostate, lung, pancreas or colon cancer cell during necrosis but be not released from a healthy cell either during apoptosis, necrosis or both.

### Chimeric Antigen Receptors (CAR)

The cell of the present invention may also comprise one or more chimeric antigen receptor(s). The CAR(s) may be specific for a tumour-associated antigen.

Classical CARs are chimeric type I trans-membrane proteins which connect an extracellular antigen-recognizing domain (binder) to an intracellular signalling domain (endodomain). The binder is typically a single-chain variable fragment (scFv) derived from a monoclonal antibody (mAb), but it can be based on other formats which comprise an antibody-like or ligand-based antigen binding site. A trans-membrane domain anchors the protein in the cell membrane and connects the spacer to the endodomain.

Early CAR designs had endodomains derived from the intracellular parts of either the γ chain of the FcεR1 or CD3ζ. Consequently, these first generation receptors transmitted immunological signal 1, which was sufficient to trigger T-cell killing of cognate target cells but failed to fully activate the T-cell to proliferate and survive. To overcome this limitation, compound endodomains have been constructed: fusion of the intracellular part of a T-cell co-stimulatory molecule to that of CD3ζ results in second generation receptors which can transmit an activating and co-stimulatory signal simultaneously after antigen recognition. The co-stimulatory domain most commonly used is that of CD28. This supplies the most potent co-stimulatory signal—namely immunological signal 2, which triggers T-cell proliferation. Some receptors have also been described which include TNF receptor family endodomains, such as the closely related OX40 and 41BB which transmit survival signals. Even more potent third generation CARs have now been described which have endodomains capable of transmitting activation, proliferation and survival signals.

CAR-encoding nucleic acids may be transferred to T cells using, for example, retroviral vectors. In this way, a large number of antigen-specific T cells can be generated for adoptive cell transfer. When the CAR binds the target-antigen, this results in the transmission of an activating signal to the T-cell it is expressed on. Thus the CAR directs the specificity and cytotoxicity of the T cell towards cells expressing the targeted antigen.

The cell of the present invention may comprise one or more CAR(s).

The CAR(s) may comprise an antigen-binding domain, a spacer domain, a transmembrane domain and an endodomain. The endodomain may comprise or associate with a domain which transmit T-cell activation signals.

### CAR Antigen Binding Domain

The antigen-binding domain is the portion of a CAR which recognizes antigen.

Numerous antigen-binding domains are known in the art, including those based on the antigen binding site of an antibody, antibody mimetics, and T-cell receptors. For example, the antigen-binding domain may comprise: a single-chain variable fragment (scFv) derived from a monoclonal antibody; a natural ligand of the target antigen; a peptide with sufficient affinity for the target; a single domain binder such as a camelid; an artificial binder single as a Darpin; or a single-chain derived from a T-cell receptor.

The term “ligand” is used synonymously with “antigen” to mean an entity which is specifically recognised and bound by the antigen-binding domain of a CAR.

### Cell Surface Antigen

The CAR may recognise a cell-surface antigen, i.e. an entity, such as a transmembrane protein which is expressed on the surface of a target cell, such as a tumour cell.

The CAR may specifically bind a tumour-associated cell-surface antigen.

Various tumour associated antigens (TAA) are known, some of which are shown in Table 1. The antigen-binding domain used in the present invention may be a domain which is capable of binding a TAA as indicated therein.

TABLE 1

| Cancer type | TAA |
|---|---|
| Diffuse Large B-cell Lymphoma | CD19, CD20, CD22 |
| Breast cancer | ErbB2, MUC1 |
| AML | CD13, CD33 |
| Neuroblastoma | GD2, NCAM, ALK, GD2 |
| B-CLL | CD19, CD52, CD160 |
| Colorectal cancer | Folate binding protein, CA-125 |
| Chronic Lymphocytic Leukaemia | CD5, CD19 |
| Glioma | EGFR, Vimentin |
| Multiple myeloma | BCMA, CD138 |
| Renal Cell Carcinoma | Carbonic anhydrase IX, G250 |
| Prostate cancer | PSMA |
| Bowel cancer | A33 |

Where the CAR recognises a B-cell lymphoma or leukemia antigen (such as CD19, CD20, CD52, CD160 or CD5), the CCR may recognise another B-cell antigen, such as CD22.

### Prostate-Cancer Associated Antigens

The CAR may specifically bind a cell-surface antigen associated with prostate cancer, such as prostate stem cell antigen (PSCA) or prostate-specific membrane antigen (PSMA).

PSCA is a glycosylphosphatidylinositol-anchored cell membrane glycoprotein. It is up-regulated in a large proportion of prostate cancers and is also detected in cancers of the bladder and pancreas.

Various anti-PSCA antibodies are known, such as 7F5 (Morgenroth et al (Prostate (2007) 67:1121-1131); 1G8 (Hillerdal et al (2014) BMC Cancer 14:30); and Ha1-4.117 (Abate-Daga et al (2014) 25:1003-1012).

The CCR-expressing cell of the invention may also express an anti-PSCA CAR which may comprise an antigen binding domain based on one of these antibodies.

PSMA is a zinc metalloenzyme that resides in membranes. PSMA is strongly expressed in the human prostate, being a hundredfold greater than the expression in most other tissues. In cancer, it is upregulated in expression and has been called the second-most-upregulated gene in prostate cancer, with increase of 8- to 12-fold over the noncancerous prostate. In addition to the expression in the human prostate and prostate cancer, PSMA is also found to be highly expressed in tumor neovasculature but not normal vasculature of all types of solid tumors, such as kidney, breast, colon, etc.

Various anti-PSMA antibodies are known, such as 7E11, J591, J415, and Hybritech PEQ226.5 and PM2J004.5 each of which binds a distinct epitope of PSMA (Chang et al (1999) Cancer Res 15:3192-8).

The CCR-expressing cell of the invention may also express an anti-PSMA CAR which may comprise an antigen binding domain based on one of these antibodies.

For example, the CAR may comprise an scFv based on J591, having the sequence shown as SEQ ID No. 11.

(J591 scFv)
SEQ ID No. 11
EVQLQQSGPELKKPGTSVRISCKTSGYTFTEYTIHVVVKQSHGKSL
EWIGNINPNNGGTTYNQKFEDKATLTVDKSSSTAYMELRSLTSEDS
AVYYCAAGWNFDYWGQGTTLTVSSGGGGSGGGGSGGGGSDIVMTQS
HKFMSTSVGDRVSIICKASQDVGTAVDWYQQKPGQSPKWYWASTRH
TGVPDRFTGSGSGTDFTLTITNVQSEDLADYFCQQYNSYPLTFGAG
TMLDLKR

### CAR Transmembrane Domain

The transmembrane domain is the sequence of a CAR that spans the membrane. It may comprise a hydrophobic alpha helix. The CAR transmembrane domain may be derived from CD28, which gives good receptor stability.

### CAR Signal Peptide

The CAR and CCR described herein may comprise a signal peptide so that when it/they is expressed in a cell, such as a T-cell, the nascent protein is directed to the endoplasmic reticulum and subsequently to the cell surface, where it is expressed.

The core of the signal peptide may contain a long stretch of hydrophobic amino acids that has a tendency to form a single alpha-helix. The signal peptide may begin with a short positively charged stretch of amino acids, which helps to enforce proper topology of the polypeptide during translocation. At the end of the signal peptide there is typically a stretch of amino acids that is recognized and cleaved by signal peptidase. Signal peptidase may cleave either during or after completion of translocation to generate a free signal peptide and a mature protein. The free signal peptides are then digested by specific proteases.

The signal peptide may be at the amino terminus of the molecule.

The signal peptide may comprise the sequence shown as SEQ ID No. 12, 13 or 14 or a variant thereof having 5, 4, 3, 2 or 1 amino acid mutations (insertions, substitutions or additions) provided that the signal peptide still functions to cause cell surface expression of the CAR.

SEQ ID No. 12:
MGTSLLCWMALCLLGADHADG

The signal peptide of SEQ ID No. 12 is compact and highly efficient and is derived from TCR beta chain. It is predicted to give about 95% cleavage after the terminal glycine, giving efficient removal by signal peptidase.

SEQ ID No. 13:
MSLPVTALLLPLALLLHAARP

The signal peptide of SEQ ID No. 13 is derived from IgG1.

SEQ ID No. 14:
MAVPTQVLGLLLLWLTDARC

The signal peptide of SEQ ID No. 14 is derived from CD8a.

### CAR Endodomain

The endodomain is the portion of a classical CAR which is located on the intracellular side of the membrane.

The endodomain is the signal-transmission portion of a classical CAR. After antigen recognition by the antigen binding domain, individual CAR molecules cluster, native CD45 and CD148 are excluded from the synapse and a signal is transmitted to the cell.

The CAR endodomain may be or comprise an intracellular signalling domain. In an alternative embodiment, the endodomain of the present CAR may be capable of interacting with an intracellular signalling molecule which is present in the cytoplasm, leading to signalling.

The intracellular signalling domain or separate intracellular signalling molecule may be or comprise a T cell signalling domain.

The most commonly used signalling domain component is that of CD3-zeta endodomain, which contains 3 ITAMs. This transmits an activation signal to the T cell after antigen is bound. CD3-zeta may not provide a fully competent activation signal and additional co-stimulatory signalling may be needed. For example, chimeric CD28 and OX40 can be used with CD3-Zeta to transmit a proliferative/survival signal, or all three can be used together.

The CAR may comprise the CD3-Zeta endodomain alone, the CD3-Zeta endodomain with that of either CD28 or OX40 or the CD28 endodomain and OX40 and CD3-Zeta endodomain.

The CAR endodomain may comprise one or more of the following: an ICOS endodomain, a CD27 endodomain, a BTLA endodomain, a CD30 endodomain, a GITR endodomain and an HVEM endodomain.

The endomain may comprise the sequence shown as SEQ ID No. 15 to 23 or a variant thereof having at least 80% sequence identity.

CD3 Z endodomain
SEQ ID No. 15
RVKFSRSADAPAYQQGQNQLYNELNLGRREEYDVLDKRRGRDPEMGGK
PRRKNPQEGLYNELQKDKMAEAYSEIGMKGERRRGKGHDGLYQGLSTA
TKDTYDALHMQALPPR
CD28 and CD3 Zeta endodomains
SEQ ID No. 16
SKRSRLLHSDYMNMTPRRPGPTRKHYQPYAPPRDFAAYRSRVKFSRSA
DAPAYQQGQNQLYNELNLGRREEYDVLDKRRGRDPEMGGKPRRKNPQE
GLYNELQKDKMAEAYSEIGMKGERRRGKGHDGLYQGLSTATKDTYDAL
HMQALPPR
CD28, OX40 and CD3 Zeta endodomains
SEQ ID No. 17
SKRSRLLHSDYMNMTPRRPGPTRKHYQPYAPPRDFAAYRSRDQRLPPD
AHKPPGGGSFRTPIQEEQADAHSTLAKIRVKFSRSADAPAYQQGQNQL
YNELNLGRREEYDVLDKRRGRDPEMGGKPRRKNPQEGLYNELQKDKMA
EAYSEIGMKGERRRGKGHDGLYQGLSTATKDTYDALHMQALPPR
ICOS endodomain
SEQ ID No. 18
CWLTKKKYSSSVHDPNGEYMFMRAVNTAKKSRLTDVTL
CD27 endodomain
SEQ ID No. 19
QRRKYRSNKGESPVEPAEPCHYSCPREEEGSTIPIQEDYRKPEPACSP
BTLA endodomain
SEQ ID No. 20
RRHQGKQNELSDTAGREINLVDAHLKSEQTEASTRQNSQVLLSETGIY
DNDPDLCFRMQEGSEVYSNPCLEENKPGIVYASLNHSVIGPNSRLARN
VKEAPTEYASICVRS
CD30 endodomain
SEQ ID No. 21
HRRACRKRIRQKLHLCYPVQTSQPKLELVDSRPRRSSTQLRSGASVT
EPVAEERGLMSQPLMETCHSVGAAYLESLPLQDASPAGGPSSPRDLPE
PRVSTEHTNNKIEKIYIMKADTVIVGTVKAELPEGRGLAGPAEPELEE
ELEADHTPHYPEQETEPPLGSCSDVMLSVEEEGKEDPLPTAASGK
GITR endodomain
SEQ ID No. 22
QLGLHIWQLRSQCMWPRETQLLLEVPPSTEDARSCQFPEEERGERSAE
EKGRLGDLVVV
HVEM endodomain
SEQ ID No. 23
CVKRRKPRGDVVKVIVSVQRKRQEAEGEATVIEALQAPPDVTTVAVEE
TIPSFTGRSPNH

A variant sequence may have at least 80%, 85%, 90%, 95%, 98% or 99% sequence identity to SEQ ID No. 15 to 23, provided that the sequence provides an effective intracellular signalling domain.

### Nucleic Acid

The present invention also provides a nucleic acid encoding a CCR of the invention.

The nucleic acid may have the structure:

### AgB-spacer-TM-endo

in which
AgB1 is a nucleic acid sequence encoding the antigen-binding domain of the CCR; spacer 1 is a nucleic acid sequence encoding the spacer of the CCR;
TM1 is a nucleic acid sequence encoding the transmembrane domain of the CCR;
endo 1 is a nucleic acid sequence encoding the endodomain of the CCR.

### Nucleic Acid Construct

The present invention further provides a nucleic acid construct which comprises a first nucleic acid sequence encoding a first CCR as defined in connection with the first aspect of the invention; and a second nucleic acid sequence encoding a second CCR as defined in connection with the first aspect of the invention.

The nucleic acid construct may have the following structure:

AgB1-spacer1-TM1-endo1-coexpr-AgB2-spacer2-TM2-endo2
in which
AgB1 is a nucleic acid sequence encoding the antigen-binding domain of the first CCR;
spacer 1 is a nucleic acid sequence encoding the spacer of the first CCR;
TM1 is a nucleic acid sequence encoding the transmembrane domain of the first CCR;
endo 1 is a nucleic acid sequence encoding the endodomain of the first CCR;
coexpr is a nucleic acid sequence enabling co-expression of both CCRs
AgB2 is a nucleic acid sequence encoding the antigen-binding domain of the second CCR;
spacer 2 is a nucleic acid sequence encoding the spacer of the second CCR;
TM2 is a nucleic acid sequence encoding the transmembrane domain of the second CCR;
endo 2 is a nucleic acid sequence encoding the endodomain of the second CCR.

When the nucleic acid construct is expressed in a cell, such as a T-cell, it encodes a polypeptide which is cleaved at the cleavage site such that the first and second CCRs are co-expressed at the cell surface.

The first and second CCRs may bind distinct epitopes on the same antigen.

The first and second CCRs may have complementary endodomains e.g. one derived from the α or β chain of a cytokine receptor and one derived from the γ chain of the same cytokine receptor.

The present invention also provides a nucleic acid construct encoding a CCR of the invention and a CAR. Such a construct may have the structure:

### CCRAgB-CCRspacer-CCRTM-CCRendo-coexpr-CARAgB-CARspacer-CARTM-CARendo

or

### CARAgB-CARspacer-CARTM-CARendo-coexpr-CCRAgB-CCRspacer-CCRTM-CCRendo

in which
CCRAgB is a nucleic acid sequence encoding the antigen-binding domain of the CCR;
CCRspacer is a nucleic acid sequence encoding the spacer of the CCR;
CCRTM is a nucleic acid sequence encoding the transmembrane domain of the CCR;
CCRendo is a nucleic acid sequence encoding the endodomain of the CCR;
coexpr is a nucleic acid sequence enabling co-expression of both the CCR and the CAR
CARAgB is a nucleic acid sequence encoding the antigen-binding domain of the CAR;
CARspacer is a nucleic acid sequence encoding the spacer of the CAR;
CARTM is a nucleic acid sequence encoding the transmembrane domain of the CAR; and
CARendo is a nucleic acid sequence encoding the endodomain of the CAR.

The present invention also provides a nucleic acid construct encoding a first and a second CCR of the invention and a CAR. The first and second CCRs may bind separate epitopes on the same antigen. Such a construct may have the structure:

(i) CCRAgB1-CCRspacer1-CCRTM1-CCRendo1-coexpr1-CCRAgB2-CCRspacer2-CCRTM2-CCRendo2-coexpr2-CARAgB-CARspacer-CARTM-CARendo;
(ii) CCRAgB1-CCRspacer1-CCRTM1-CCRendo1-coexpr1-CARAgB-CARspacer-CARTM-CARendo-coexpr2-CCRAgB2-CCRspacer2-CCRTM2-CCRendo2; or
(iii) CARAgB-CARspacer-CARTM-CARendo-coexpr1-CCRAgB1-CCRspacer1-CCRTM1-CCRendo1-coexpr2-CCRAgB2-CCRspacer2-CCRTM2-CCRendo2;
in which
CCRAgB1 is a nucleic acid sequence encoding the antigen-binding domain of the first CCR;
CCRspacer1 is a nucleic acid sequence encoding the spacer of the first CCR;
CCRTM1 is a nucleic acid sequence encoding the transmembrane domain of the first CCR;
CCRendo1 is a nucleic acid sequence encoding the endodomain of the first CCR;
CCRAgB2 is a nucleic acid sequence encoding the antigen-binding domain of the second CCR;
CCRspacer2 is a nucleic acid sequence encoding the spacer of the second CCR;
CCRTM2 is a nucleic acid sequence encoding the transmembrane domain of the second CCR;
CCRendo2 is a nucleic acid sequence encoding the endodomain of the second CCR;
Coexpr1 and coexpr2 are nucleic acid sequences enabling co-expression of the two flanking sequences;
CARAgB is a nucleic acid sequence encoding the antigen-binding domain of the CAR;
CARspacer is a nucleic acid sequence encoding the spacer of the CAR;
CARTM is a nucleic acid sequence encoding the transmembrane domain of the CAR; and
CARendo is a nucleic acid sequence encoding the endodomain of the CAR.

As used herein, the terms “polynucleotide”, “nucleotide”, and “nucleic acid” are intended to be synonymous with each other.

It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described here to reflect the codon usage of any particular host organism in which the polypeptides are to be expressed.

Nucleic acids according to the invention may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the use as described herein, it is to be understood that the polynucleotides may be modified by any method available in the art. Such modifications may be carried out in order to enhance the in vivo activity or life span of polynucleotides of interest.

The terms “variant”, “homologue” or “derivative” in relation to a nucleotide sequence include any substitution of, variation of, modification of, replacement of, deletion of or addition of one (or more) nucleic acid from or to the sequence.

In the structure above, “coexpr” is a nucleic acid sequence enabling co-expression of both first and second CARs. It may be a sequence encoding a cleavage site, such that the nucleic acid construct produces comprises two or more CCRs, or a CCR and a CAR, joined by a cleavage site(s). The cleavage site may be self-cleaving, such that when the polypeptide is produced, it is immediately cleaved into individual peptides without the need for any external cleavage activity.

The cleavage site may be any sequence which enables the first and second CCRs, or CCR and CAR, to become separated.

The term “cleavage” is used herein for convenience, but the cleavage site may cause the peptides to separate into individual entities by a mechanism other than classical cleavage. For example, for the Foot-and-Mouth disease virus (FMDV) 2A self-cleaving peptide (see below), various models have been proposed for to account for the “cleavage” activity: proteolysis by a host-cell proteinase, autoproteolysis or a translational effect (Donnelly et al (2001) J. Gen. Virol. 82:1027-1041). The exact mechanism of such “cleavage” is not important for the purposes of the present invention, as long as the cleavage site, when positioned between nucleic acid sequences which encode proteins, causes the proteins to be expressed as separate entities.

The cleavage site may be a furin cleavage site.

Furin is an enzyme which belongs to the subtilisin-like proprotein convertase family. The members of this family are proprotein convertases that process latent precursor proteins into their biologically active products. Furin is a calcium-dependent serine endoprotease that can efficiently cleave precursor proteins at their paired basic amino acid processing sites. Examples of furin substrates include proparathyroid hormone, transforming growth factor beta 1 precursor, proalbumin, pro-beta-secretase, membrane type-1 matrix metalloproteinase, beta subunit of pro-nerve growth factor and von Willebrand factor. Furin cleaves proteins just downstream of a basic amino acid target sequence (canonically, Arg-X-(Arg/Lys)-Arg′) and is enriched in the Golgi apparatus.

The cleavage site may be a Tobacco Etch Virus (TEV) cleavage site.

TEV protease is a highly sequence-specific cysteine protease which is chymotrypsin-like proteases. It is very specific for its target cleavage site and is therefore frequently used for the controlled cleavage of fusion proteins both in vitro and in vivo. The consensus TEV cleavage site is ENLYFQ\S (where ‘\’ denotes the cleaved peptide bond). Mammalian cells, such as human cells, do not express TEV protease. Thus in embodiments in which the present nucleic acid construct comprises a TEV cleavage site and is expressed in a mammalian cell—exogenous TEV protease must also expressed in the mammalian cell.

The cleavage site may encode a self-cleaving peptide.

A ‘self-cleaving peptide’ refers to a peptide which functions such that when the polypeptide comprising the proteins and the self-cleaving peptide is produced, it is immediately “cleaved” or separated into distinct and discrete first and second polypeptides without the need for any external cleavage activity.

The self-cleaving peptide may be a 2A self-cleaving peptide from an aphtho- or a cardiovirus. The primary 2A/2B cleavage of the aptho- and cardioviruses is mediated by 2A “cleaving” at its own C-terminus. In apthoviruses, such as foot-and-mouth disease viruses (FMDV) and equine rhinitis A virus, the 2A region is a short section of about 18 amino acids, which, together with the N-terminal residue of protein 2B (a conserved proline residue) represents an autonomous element capable of mediating “cleavage” at its own C-terminus (Donelly et al (2001) as above).

“2A-like” sequences have been found in picornaviruses other than aptho- or cardioviruses, ‘picornavirus-like’ insect viruses, type C rotaviruses and repeated sequences within Trypanosoma spp and a bacterial sequence (Donnelly et al (2001) as above). The cleavage site may comprise one of these 2A-like sequences, such as:

(SEQ ID No. 24)
YHADYYKQRLIHDVEMNPGP
(SEQ ID No. 25)
HYAGYFADLLIHDIETNPGP
(SEQ ID No. 26)
QCTNYALLKLAGDVESNPGP
(SEQ ID No. 27)
ATNFSLLKQAGDVEENPGP
(SEQ ID No. 28)
AARQMLLLLSGDVETNPGP
(SEQ ID No. 29)
RAEGRGSLLTCGDVEENPGP
(SEQ ID No. 30)
TRAEIEDELIRAGIESNPGP
(SEQ ID No. 31)
TRAEIEDELIRADIESNPGP
(SEQ ID No. 32)
AKFQIDKILISGDVELNPGP
(SEQ ID No. 33)
SSIIRTKMLVSGDVEENPGP
(SEQ ID No. 34)
CDAQRQKLLLSGDIEQNPGP
(SEQ ID No. 35)
YPIDFGGFLVKADSEFNPGP

The cleavage site may comprise the 2A-like sequence shown as SEQ ID No. 29

(RAEGRGSLLTCGDVEENPGP).

The present invention also provides a kit comprising one or more nucleic acid sequence(s) encoding first and second CCRs according to the first aspect of the present invention, or one or more CCR(s) according to the invention and one or more CAR(s).

### Vector

The present invention also provides a vector, or kit of vectors, which comprises one or more nucleic acid sequence(s) encoding a one or more CCR(s) according to the first aspect of the invention and optionally one or more CAR(s). Such a vector may be used to introduce the nucleic acid sequence(s) into a host cell so that it expresses a CCR according to the first aspect of the invention.

The vector may, for example, be a plasmid or a viral vector, such as a retroviral vector or a lentiviral vector, or a transposon based vector or synthetic mRNA.

The vector may be capable of transfecting or transducing a T cell or a NK cell.

### Cell

The present invention provides a cell which comprises one or more CCR(s) of the invention and optionally one of more CAR(s).

The cell may comprise a nucleic acid or a vector of the present invention.

The cell may be a cytolytic immune cell such as a T cell or an NK cell.

T cells or T lymphocytes are a type of lymphocyte that play a central role in cell-mediated immunity. They can be distinguished from other lymphocytes, such as B cells and natural killer cells (NK cells), by the presence of a T-cell receptor (TCR) on the cell surface. There are various types of T cell, as summarised below.

Helper T helper cells (TH cells) assist other white blood cells in immunologic processes, including maturation of B cells into plasma cells and memory B cells, and activation of cytotoxic T cells and macrophages. TH cells express CD4 on their surface. TH cells become activated when they are presented with peptide antigens by MHC class II molecules on the surface of antigen presenting cells (APCs). These cells can differentiate into one of several subtypes, including TH1, TH2, TH3, TH17, Th9, or TFH, which secrete different cytokines to facilitate different types of immune responses.

Cytolytic T cells (TC cells, or CTLs) destroy virally infected cells and tumor cells, and are also implicated in transplant rejection. CTLs express the CD8 at their surface. These cells recognize their targets by binding to antigen associated with MHC class I, which is present on the surface of all nucleated cells. Through IL-10, adenosine and other molecules secreted by regulatory T cells, the CD8+ cells can be inactivated to an anergic state, which prevent autoimmune diseases such as experimental autoimmune encephalomyelitis.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory T cells comprise three subtypes: central memory T cells (TCM cells) and two types of effector memory T cells (TEM cells and TEMRA cells). Memory cells may be either CD4+ or CD8+. Memory T cells typically express the cell surface protein CD45RO.

Regulatory T cells (Treg cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus.

Two major classes of CD4+ Treg cells have been described—naturally occurring Treg cells and adaptive Treg cells.

Naturally occurring Treg cells (also known as CD4+CD25+FoxP3+ Treg cells) arise in the thymus and have been linked to interactions between developing T cells with both myeloid (CD11c+) and plasmacytoid (CD123+) dendritic cells that have been activated with TSLP. Naturally occurring Treg cells can be distinguished from other T cells by the presence of an intracellular molecule called FoxP3. Mutations of the FOXP3 gene can prevent regulatory T cell development, causing the fatal autoimmune disease IPEX.

Adaptive Treg cells (also known as Tr1 cells or Th3 cells) may originate during a normal immune response.

The cell may be a Natural Killer cell (or NK cell). NK cells form part of the innate immune system. NK cells provide rapid responses to innate signals from virally infected cells in an MHC independent manner

NK cells (belonging to the group of innate lymphoid cells) are defined as large granular lymphocytes (LGL) and constitute the third kind of cells differentiated from the common lymphoid progenitor generating B and T lymphocytes. NK cells are known to differentiate and mature in the bone marrow, lymph node, spleen, tonsils and thymus where they then enter into the circulation.

The CCR-expressing cells of the invention may be any of the cell types mentioned above.

T or NK cells according to the first aspect of the invention may either be created ex vivo either from a patient's own peripheral blood (1st party), or in the setting of a haematopoietic stem cell transplant from donor peripheral blood (2nd party), or peripheral blood from an unconnected donor (3rd party).

Alternatively, T or NK cells according to the first aspect of the invention may be derived from ex vivo differentiation of inducible progenitor cells or embryonic progenitor cells to T or NK cells. Alternatively, an immortalized T-cell line which retains its lytic function and could act as a therapeutic may be used.

In all these embodiments, CCR-expressing cells are generated by introducing DNA or RNA coding for the or each CCR(s) by one of many means including transduction with a viral vector, transfection with DNA or RNA.

The cell of the invention may be an ex vivo T or NK cell from a subject. The T or NK cell may be from a peripheral blood mononuclear cell (PBMC) sample. T or NK cells may be activated and/or expanded prior to being transduced with nucleic acid encoding the molecules providing the CCR according to the first aspect of the invention, for example by treatment with an anti-CD3 monoclonal antibody.

The T or NK cell of the invention may be made by:
(i) isolation of a T or NK cell-containing sample from a subject or other sources listed above; and

(ii) transduction or transfection of the T or NK cells with one or more a nucleic acid sequence(s) encoding a CCR.

The T or NK cells may then by purified, for example, selected on the basis of expression of the antigen-binding domain of the antigen-binding polypeptide.

### Pharmaceutical Composition

The present invention also relates to a pharmaceutical composition containing a plurality of cells according to the invention.

The pharmaceutical composition may additionally comprise a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

### Method of Treatment

The present invention provides a method for treating a disease which comprises the step of administering the cells of the present invention (for example in a pharmaceutical composition as described above) to a subject.

A method for treating a disease relates to the therapeutic use of the cells of the present invention. Herein the cells may be administered to a subject having an existing disease or condition in order to lessen, reduce or improve at least one symptom associated with the disease and/or to slow down, reduce or block the progression of the disease.

The method may involve the steps of:
(i) isolating a T or NK cell-containing sample;
(ii) transducing or transfecting such cells with a nucleic acid sequence or vector provided by the present invention;
(iii) administering the cells from (ii) to a subject.

The T or NK cell-containing sample may be isolated from a subject or from other sources, for example as described above. The T or NK cells may be isolated from a subject's own peripheral blood (1st party), or in the setting of a haematopoietic stem cell transplant from donor peripheral blood (2nd party), or peripheral blood from an unconnected donor (3rd party).

The present invention provides a CCR-expressing cell of the present invention for use in treating a disease.

The invention also relates to the use of a CCR-expressing cell of the present invention in the manufacture of a medicament for the treatment of a disease.

The disease to be treated by the methods of the present invention may be a cancerous disease, such as bladder cancer, breast cancer, colon cancer, endometrial cancer, kidney cancer (renal cell), lung cancer, melanoma, pancreatic cancer, prostate cancer and thyroid cancer.

The disease may be a solid tumour, for example a tumour of the bone, muscles or one or more organs. The solid tumour may be a sarcoma, for example a sarcoma in a blood vessel, bone, fat tissue, ligament, lymph vessel, muscle or tendon. The solid tumour may be a carcinoma, i.e. a tumour that forms in epithelial cells. Epithelial cells are found in the skin, glands and the linings of organs such as the bladder, ureters and part of the kidneys.

The disease may be a cancer of one of the following tissues: colon, prostate, breast, lung, skin, liver, bone, ovary, pancreas, brain, head or neck.

The cells of the present invention may be capable of killing target cells, such as cancer cells. The target cell may be characterised by the presence of a necrotic marker in the vicinity of the target cell. The target cell may be characterised by the presence of a necrotic marker together with the expression of a tumour-associated antigen (TAA) at the target cell surface.

The cells and pharmaceutical compositions of present invention may be for use in the treatment of the diseases described above.

The cells and pharmaceutical compositions of present invention may be for use in any of the methods described above.

### Chimeric Transmembrane Protein

The present invention also provides a chimeric transmembrane protein comprising a dimerization domain; and a cytokine receptor endodomain.

Dimerisation may occur spontaneously, in which case the chimeric transmembrane protein will be constitutively active. Alternatively, dimerization may occur only in the presence of a chemical inducer of dimerization (CID) in which case the transmembrane protein only causes cytokine-type signalling in the presence of the CID.

Suitable dimerization domains and CIDs are described in WO2015/150771, the contents of which are hereby incorporated by reference.

For example, one dimerization domain may comprise the rapamycin binding domain of FK-binding protein 12 (FKBP12), the other may comprise the FKBP12-Rapamycin Binding (FRB) domain of mTOR; and the CID may be rapamycin or a derivative thereof.

One dimerization domain may comprise the FK506 (Tacrolimus) binding domain of FK-binding protein 12 (FKBP12) and the other dimerization domain may comprise the cyclosporin binding domain of cylcophilin A; and the CID may be an FK506/cyclosporin fusion or a derivative thereof.

One dimerization domain may comprise an oestrogen-binding domain (EBD) and the other dimerization domain may comprise a streptavidin binding domain; and the CID may be an estrone/biotin fusion protein or a derivative thereof.

One dimerization domain may comprise a glucocorticoid-binding domain (GBD) and the other dimerization domain may comprise a dihydrofolate reductase (DHFR) binding domain; and the CID may be a dexamethasone/methotrexate fusion protein or a derivative thereof.

One dimerization domain may comprise an 06-alkylguanine-DNA alkyltransferase (AGT) binding domain and the other dimerization domain may comprise a dihydrofolate reductase (DHFR) binding domain; and the CID may be an 06-benzylguanine derivative/methotrexate fusion protein or a derivative thereof.

One dimerization domain may comprise a retinoic acid receptor domain and the other dimerization domain may comprise an ecodysone receptor domain; and the CID may be RSL1 or a derivative thereof.

Where the dimerization domain spontaneously heterodimerizes, it may be based on the dimerization domain of an antibody. In particular it may comprise the dimerization portion of a heavy chain constant domain (CH) and a light chain constant domain (CL). The “dimerization portion” of a constant domain is the part of the sequence which forms the inter-chain disulphide bond.

The chimeric cytokine receptor may comprise the Fab portion of an antibody as exodomain, for example as illustrated schematically in FIG. 5.

The chimeric transmembrane protein may comprise two polypeptides:
(i) a first polypeptide which comprises:
   (a) a first dimerisation domain; and
   (b) a first chain of the cytokine receptor endodomain; and
(ii) a second polypeptide which comprises:
   (a) a second dimerization domain, which dimerises with the first dimerization domain; and
   (b) a second chain of the cytokine-receptor endodomain.

The sections above defining the cytokine receptor endodomain of the chimeric cytokine receptor also apply to the chimeric transmembrane protein of the present invention.

The sections above relating to nucleic acids, vectors, kits, cells, pharmaceutical compositions and methods also apply to the chimeric transmembrane protein of the present invention.

The invention will now be further described by way of Examples, which are meant to serve to assist one of ordinary skill in the art in carrying out the invention and are not intended in any way to limit the scope of the invention.

### EXAMPLES

### Example 1—In Vitro Testing of PSA-Sensing CCR

T-cells are transduced with either a PSMA-specific CAR, or transduced with a construct which co-expresses a PSMA-specific CAR with a PSA-specific CCR. T-cells are co-cultured with PSMA expressing target cells which secrete or do not secrete PSA. This co-culture is conducted in the presence or absence of exogenous IL2. This co-culture is conducted at different effector to target ratios. This co-culture is repeated serially with T-cells challenged with repeated target cells. Proliferation of T-cells and killing of target cells is determined. In this way, the contribution to proliferation and survival of T-cells the CCR makes can be measured.

### Example 2—In Vivo Testing of PSA-Sensing CCR

NSG mice are engrafted with a human prostate cancer cell line which expresses PSMA and secretes PSA and which expresses firefly Luciferase. T-cells are transduced with either a PSMA-specific CAR, or transduced with a construct which co-expresses the PSMA-specific CAR with a PSA-specific CCR. T-cells are administered to the mice. The tumour burden can be serially measured using bioluminescent imaging and the response to CAR T-cells evaluated. Mice within each cohort can be sacrificed at different time-points and tumour burden directly measured by macroscopic measurements and by immunohistochemistry. Further, engraftment/expansion of T-cells at the tumour bed or within lymphoid tissues such as lymph nodes, spleen and bone-marrow measured by flow cytometry of said tissues.

### Example 3—Creation of and Testing a Constitutively Active Cytokine-Signalling Molecule

A constitutively active cytokine-signalling chimeric transmembrane protein was produced by linking cytokine receptor endodomains to a “Fab” type exodomain (FIG. 5). This structure uses the natural dimerization components of antibodies, namely the dimerization domain from the heavy and light chain constant regions. The chimeric transmembrane protein has two chains; a first polypeptide which comprises the antibody light K chain and the IL2 receptor common γ chain as endodomain; and a second polypeptide which comprises the antibody heavy chain CH1 and an endodomain which comprises either: the IL2 receptor β chain (giving a constitutively active IL2-signalling molecule); or the IL7 receptor (giving a constitutively active IL7-signalling molecule). The constitutively active cytokine-signalling chimeric transmembrane proteins tested in this study included the scFv heavy and light chain variable regions. These domains are not needed for dimerization to occur. The signal is independent of antigen binding and the structure could equally be “headless” (as shown in FIG. 5) or comprise another entity such as a protein tag.

Nucleic acid sequences encoding these two polypeptides were cloned in frame separated by a 2A-peptide encoding sequence.

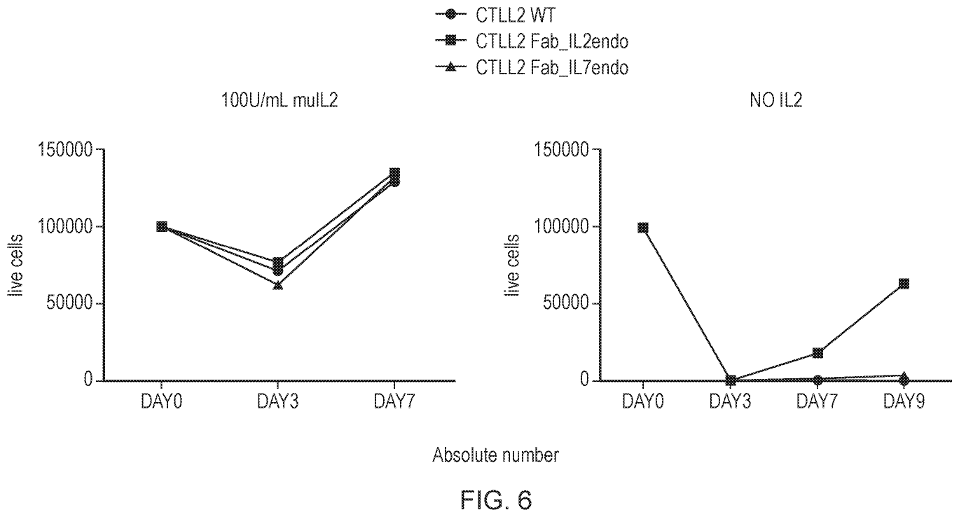

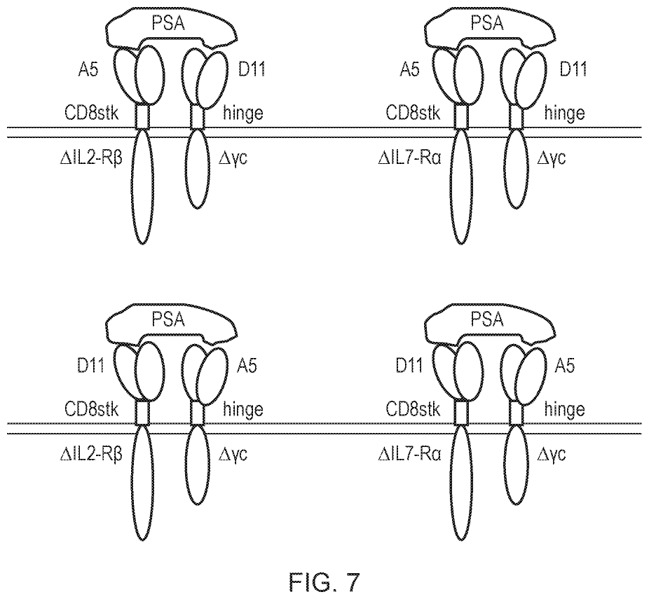

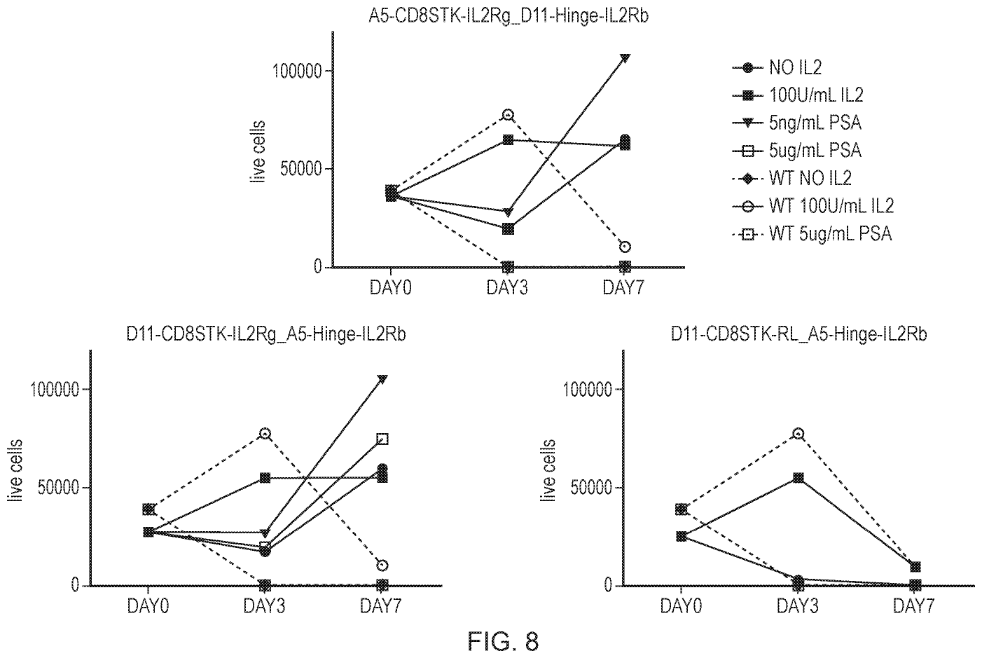

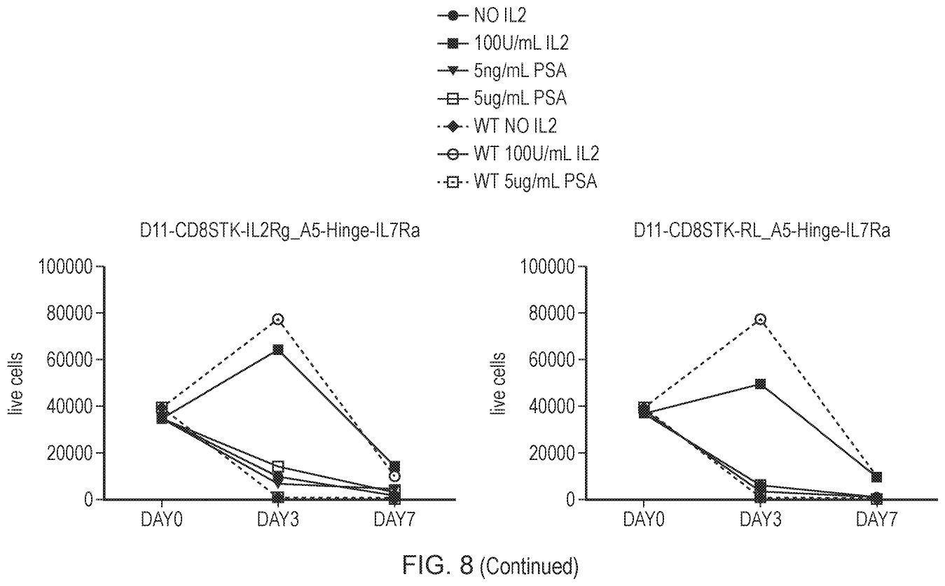

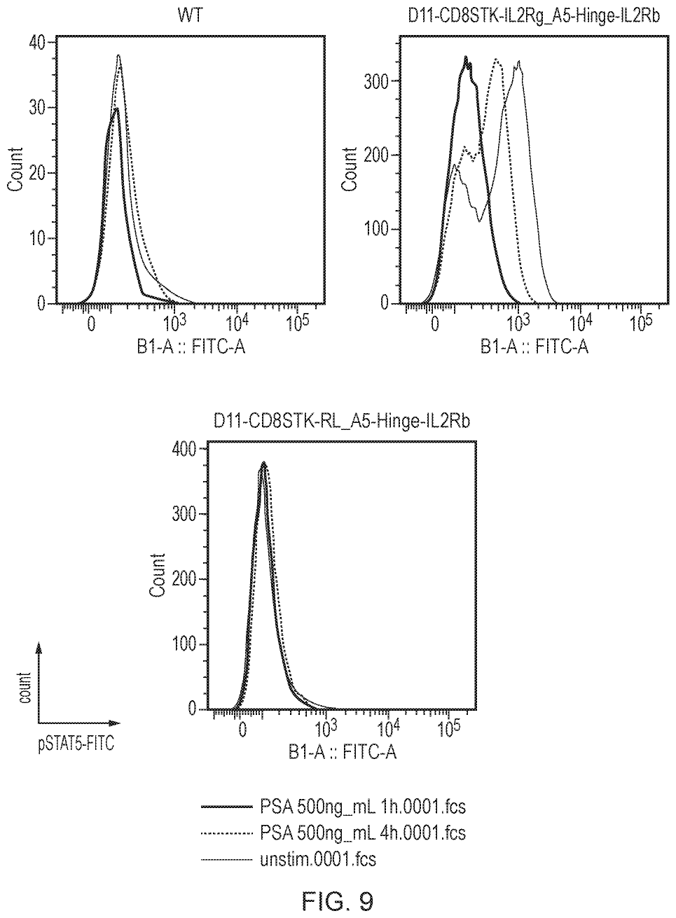

CTLL-2 (ATCC® TIB-214™) are murine cytotoxic T lymphocyte cells which are dependent upon IL-2 for growth. In the absence of IL-2 the cells undergo apoptosis. CTLL-2 cells were transduced with a vector expressing the chimeric protein comprising an IL2-receptor endodomain (Fab_IL2endo) or a vector expressing the chimeric protein comprising an IL7 receptor endodomain (Fab_IL7endo) or left untransduced (WT). As a positive control, cells of all three types were co-cultured with 100 U/ml murine IL2. Cell proliferation was assessed after 3 and 7 days of culture and the results are shown in FIG. 6.

Untransduced CTLL2 cells, together with CTLL2 cells transduced with either construct (Fab_IL2endo or Fab_IL7endo) proliferated in the presence of 100 U/mL murine IL2 (FIG. 6, left-hand panel). However, in the absence of exogenously added IL2, only cells transduced with the construct having an IL2R endodomain (Fab_IL2endo) survived and proliferated. This shows that the chimeric transmembrane receptor provides the CTLL2 cells with the necessary IL2 signal.

### Example 4—Generation and Testing of a Chimeric Cytokine Receptor Against PSA

A panel of chimeric cytokine receptors targeting PSA was developed using scFvs derived from two antibodies which bind to different PSA epitopes: 5D5A5 and 5D3D11. The crystal structure of PSA has been obtained in a sandwich complex with these two (Stura et al (2011) as above).

Schematic diagrams illustrating some of the panel of CCRs is illustrated in FIG. 7.

The panel included the following constructs:

A5-CD8stk-IL2Rg_D11-Hinge-IL2Rb: A CCR with an IL-2R endodomain having A5 on the chain with common γ chain and D11 on the chain with the IL2R β chain;
D11-CD8stk-IL2Rg_A5-Hinge-IL2Rb: A CCR with an IL-2R endodomain having D11 on the chain with common γ chain and A5 on the chain with IL2R β chain;
D11-CD8stk-RL_A5-Hinge-IL2Rb: A negative control construct which is equivalent D11-CD8stk-IL2Rg_A5-Hinge-IL2Rb, but in which the IL2Rγ chain is replaced by a rigid linker;
D11-CD8stk-IL2Rg_A5-Hinge-IL7Ra: A CCR with an IL-7R endodomain having D11 on the chain with common γ chain and A5 on the chain with IL7R α chain; and
D11-CD8stk-RL_A5-Hinge-IL7Ra: A negative control construct which is equivalent D11-CD8stk-IL2Rg_A5-Hinge-IL7Ra, but in which the IL2Rγ chain is replaced by a rigid linker;

CTLL2 cells were transduced with vectors expressing these constructs. Cells were cultured in the presence or absence of IL2 (the presence of IL2 acting as a positive control) and the presence or absence of 5 ng/mL or 5 μg/mL PSA. CTLL2 cell proliferation was assessed after 3 and 7 days and the results are shown in FIG. 8.

CTLL2 cells expressing a CCR with an IL7 endodomain did not support CTLL2 cell survival and proliferation (FIG. 8, last two panels). The presence of murine IL-2 in these cells supported CTLL2 cell growth and proliferation at day 3, but by day 7 the majority of cells had undergone apoptosis.

The anti-PSA chimeric cytokine receptors with an IL2R endodomain supported CTLL2 cell proliferation in the absence of IL2 and the presence of PSA at both 5 ng/ml and 5 μg/ml (FIG. 8, first panel), with 5 μg/ml giving greater survival and proliferation, particularly at day 7.

Both the anti-PSA chimeric cytokine receptors with an IL2R endodomain, i.e. A5-CD8stk-IL2Rg_D11-Hinge-IL2Rb and D11-CD8stk-IL2Rg_A5-Hinge-IL2Rb, indicating that the relative positioning of the two PSA-binding domains: 5D5A5 and 5D3D11, is not important for function.

Substitution of the common γ chain with a rigid linker abolished the capacity of the CCR to support CTLL2 cell survival and proliferation (FIG. 8, third panel).

As another read-out for IL2 signalling, the phosphorylation of Y694 of STAT5 was investigated using phosphoflow.

CTLL2 cells were either untransduced (WT); transduced with a PSA CCR constructs having an IL2R endodomain (D11-CD8STK-IL2Rg_A5-Hinge-IL2Rb); or transduced with an equivalent negative control construct in which the IL2Rγ chain is replaced with a rigid linker (D11-CD8STK-RL_A5-Hinge-IL2Rb). The cells were incubated overnight in the absence of exogenously added IL-2. The following day, the cells were incubated with either Pervanadate at 500 μM (a positive control which inhibits phosphatase and will lead to STAT5 phoshorylation) or 500 ng/mL PSA for 1 or 4 hours. After incubation the cells were fixed, permeabilised and analysed by flow cytometry.

The results are shown in FIG. 9. In the cells expressing the PSA CCR, the presence of PSA lead to increasing STAT5 phosphorylation with time (FIG. 9, central panel). No such increase in phosphorylation was seen with untransduced CTLL2 cells, or with CTLL2 cells transduced with an equivalent construct in which the IL2Rγ chain is replaced with a rigid linker (FIG. 9, right hand panel).

These results are consistent with the CTLL2 survival/proliferation data shown in FIG. 8 and demonstrate that a chimeric cytokine receptor against a soluble ligand (here, PSA) can be used to trigger cytokine signalling in a T-cell.

### Example 5—In Vitro Testing of LDH-Sensing CCR

T-cells are transduced with either a PSMA-specific CAR, or transduced with a construct which co-expresses a PSMA-specific CAR with a lactate dehrdrogenase (LDH)-specific CCR. T-cells are co-cultured with PSMA expressing target cells in the presence or absence of LDH. Proliferation of T-cells and killing of target cells is determined in order to investigate the capacity of the CCR to “sense” a necrotic marker such as LDH, and the contribution of the CCR to proliferation and survival of T-cells.

The above experiment is repeated, but instead of being co-cultured in the presence of absence of LDH, T cells are co-cultured with target cells in the presence or absence of the supernatant from a human prostate cancer cell line (for example PC-93) which has been induced to undergo necrosis by the addition of hydrogen peroxide.

In a further study, T cells are co-cultured with target cells in the presence or absence of the supernatant from necrotic tissue, for example from a biopsy from a solid cancer.

### Example 6—In Vivo Testing of Necrosis-Sensing CCR

NSG mice are engrafted with a human prostate cancer cell line which expresses PSMA. T-cells are transduced with either a PSMA-specific CAR, or transduced with a construct which co-expresses the PSMA-specific CAR with a LDH-specific CCR. T-cells are administered to the mice. Mice are then treated with ZD6126 by i.p. injection to induce necrosis. ZD6126 is converted in vivo to N-acetyl-colchinol, which binds to the colchicine-binding site on tubulin and causes disruption of microtubules.

The engraftment/expansion of T-cells at the tumour bed and within lymphoid tissues such as lymph nodes, spleen and bone-marrow before and 3 and 7 days after necrosis induction are measured by flow cytometry.

The tumour burden is serially measured using bioluminescent imaging and the response to CAR T-cells evaluated. Mice within each cohort are sacrificed at different time-points and tumour burden directly measured by macroscopic measurements and by immunohistochemistry.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A chimeric cytokine receptor (CCR) comprising:
an exodomain which binds to an intracellular ligand which is released from a cell as a result of necrosis; and
a cytokine receptor endodomain.

2. A chimeric cytokine receptor according to claim 1, which comprises two polypeptides:
(i) a first polypeptide which comprises:
(a) a first antigen-binding domain which binds a first epitope of the ligand
(b) a first chain of the cytokine receptor endodomain; and
(ii) a second polypeptide which comprises:
(a) a second antigen-binding domain which binds a second epitope of the ligand
(b) a second chain of the cytokine-receptor endodomain.

3-4. (canceled)

5. A chimeric cytokine receptor according to claim 1 which comprises two polypeptides:
(i) a first polypeptide which comprises:
(a) a heavy chain variable domain (V_{H})
(b) a first chain of the cytokine receptor endodomain; and
(ii) a second polypeptide which comprises:
(a) a light chain variable domain (V_{L})
(b) a second chain of the cytokine-receptor endodomain.

6-7. (canceled)

8. A chimeric cytokine receptor according to claim 1, wherein the cytokine receptor endodomain comprises:
(i) IL-2 receptor β-chain endodomain
(ii) IL-7 receptor α-chain endodomain; or
(iii) IL-15 receptor α-chain endodomain; and/or
(iv) common γ-chain receptor endodomain.

9. A chimeric cytokine receptor according to claim 1, wherein the ligand is a cytosolic or mitochondrial protein, or the ligand is double-stranded DNA.

10. (canceled)

11. A cell which comprises a chimeric cytokine receptor according to claim 1.

12. A cell according to claim 11 which comprises a first chimeric cytokine receptor and a second chimeric cytokine receptor which bind different epitopes on the same ligand.

13. A cell according to claim 12, wherein the first chimeric cytokine receptor comprises a type I cytokine receptor endodomain α- or β-chain, and the second chimeric cytokine receptor comprises a type I cytokine receptor endodomain γ-chain, such that when the first chimeric cytokine receptor and the second cytokine receptor bind the ligand, combined signalling through the α-/β-chain and γ-chain occurs.

14. A cell according to claim 11, which also comprises a chimeric antigen receptor.

15-17. (canceled)

18. A nucleic acid sequence encoding a chimeric cytokine receptor (CCR) according to claim 1.

19. A nucleic acid construct which comprises a first nucleic acid sequence encoding a first CCR and a second nucleic acid sequence encoding a second CCR, the nucleic acid construct having the structure:
AgB1-spacer1-TM1-endo1-coexpr-AbB2-spacer2-TM2-endo2
in which
AgB1 is a nucleic acid sequence encoding the antigen-binding domain of the first CCR;
spacer 1 is a nucleic acid sequence encoding the spacer of the first CCR;
TM1 is a nucleic acid sequence encoding the transmembrane domain of the first CCR;
endo 1 is a nucleic acid sequence encoding the endodomain of the first CCR;
coexpr is a nucleic acid sequence enabling co-expression of both CCRs
AgB2 is a nucleic acid sequence encoding the antigen-binding domain of the second CCR;
spacer 2 is a nucleic acid sequence encoding the spacer of the second CCR;
TM2 is a nucleic acid sequence encoding the transmembrane domain of the second CCR;
endo 2 is a nucleic acid sequence encoding the endodomain of the second CCR.

20. A nucleic acid construct according to claim 19 which also encodes a chimeric antigen receptor (CAR), the nucleic acid construct having the structure:
(i) CCRAgB1-CCRspacer1-CCRTM1-CCRendo1-coexpr1-CCRAgB2-CCRspacer2-CCRTM2-CCRendo2-coexpr2-CARAgB-CARspacer-CARTM-CARendo;
(ii) CCRAgB1-CCRspacer1-CCRTM1-CCRendo1-coexpr1-CARAgB-CARspacer-CARTM-CARendo-coexpr2-CCRAgB2-CCRspacer2-CCRTM2-CCRendo2; or
(iii) CARAgB-CARspacer-CARTM-CARendo-coexpr1-CCRAgB1-CCRspacer1-CCRTM1-CCRendo1-coexpr2-CCRAgB2-CCRspacer2-CCRTM2-CCRendo2;
in which
CCRAgB1 is a nucleic acid sequence encoding the antigen-binding domain of the first CCR;
CCRspacer1 is a nucleic acid sequence encoding the spacer of the first CCR;
CCRTM1 is a nucleic acid sequence encoding the transmembrane domain of the first CCR;
CCRendo1 is a nucleic acid sequence encoding the endodomain of the first CCR;
CCRAgB2 is a nucleic acid sequence encoding the antigen-binding domain of the second CCR;
CCRspacer2 is a nucleic acid sequence encoding the spacer of the second CCR;
CCRTM2 is a nucleic acid sequence encoding the transmembrane domain of the second CCR;
CCRendo2 is a nucleic acid sequence encoding the endodomain of the second CCR;
Coexpr1 and coexpr2 are nucleic acid sequences enabling co-expression of the two flanking sequences;
CARAgB is a nucleic acid sequence encoding the antigen-binding domain of the CAR;
CARspacer is a nucleic acid sequence encoding the spacer of the CAR;
CARTM is a nucleic acid sequence encoding the transmembrane domain of the CAR; and
CARendo is a nucleic acid sequence encoding the endodomain of the CAR.

21-22. (canceled)

23. A vector comprising a nucleic acid construct according to claim 19.

24-26. (canceled)

27. A kit which comprises:
i) a vector comprising a nucleic acid sequence encoding a CCR
as defined in claim 1; and
ii) a vector comprising a nucleic acid sequence encoding a chimeric antigen receptor.

28. A method for making a cell according to claim 11, which comprises the step of introducing: a nucleic acid sequence according to claim 18; a nucleic acid construct according to claim 19; a vector according to claim 23; or a kit of vectors according to claim 27, into a cell.

29. (canceled)

30. A pharmaceutical composition comprising a plurality of cells according to claim 11.

31. A method for treating a disease, which comprises the step of administering a pharmaceutical composition according to claim 30 to a subject.

32. A method according to claim 31, which comprises the following steps:
(i) isolation of a cell-containing sample from a subject;
(ii) transduction or transfection of the cells with: a nucleic acid sequence according to claim 18; a nucleic acid construct according to claim 19; a vector according to claim 23; or a kit of vectors according to claim 27; and
(iii) administering the cells from (ii) to a the subject.

33-34. (canceled)

35. A method according to claim 31, wherein the disease is a solid cancer.

36. A method according to claim 35, wherein the cancer is one of the following: breast, prostate, lung, pancreas or colon cancer.

37-38. (canceled)
